(19) United States
(12) Patent Application Publication
(10) Pub. No.: US 2010/0150930 A1
(43) Pub. Date: Jun. 17, 2010

(54) MAGE-11 AS A MARKER FOR ENDOMETRIAL RECEPTIVITY TO EMBRYO TRANSPLANTATION AND A MARKER AND THERAPEUTIC TARGET IN CASTRATION-RECURRENT PROSTATE CANCER

(75) Inventors: Elizabeth M. Wilson, Chapel Hill, NC (US); Suxia Bai, Chapel Hill, NC (US); Steven L. Young, Durham, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The University of North Carolina at Chapel Hill, Chapel Hill, NC (US)

(21) Appl. No.: 12/524,388

(22) PCT Filed: Jan. 24, 2008
(86) PCT No.: PCT/US08/51899

§ 371 Date: Jan. 13, 2010

Related U.S. Application Data
(60) Provisional application No. 60/897,690, filed on Jan. 26, 2007.
(60) Provisional application No. 60/917,382, filed on May 11, 2007.

(51) Int. Cl. A61K 39/395; A61K 39/00; C12Q 1/68; G01N 33/53; A61K 38/17; A61K 31/7088; C07K 16/18; A61B 17/435

(52) U.S. Cl. 424/139.1; 424/185.1; 435/6; 435/7.1; 514/12; 514/44 R; 514/44 A; 530/387.9; 600/34

(57) ABSTRACT

Compositions and methods for determining endometrial receptivity to embryo implantation and for detecting and treating castration-recurrent prostate cancer are provided. The methods comprise measuring the level of melanoma antigen gene protein-11 (MAGE-11, also referred to as MAGE-Al 1) in an endometrial or prostate tissue sample. The level of MAGE-11 protein or mRNA can be correlated to endometrial receptivity to embryo implantation in a female human or nonhuman primate, or to the presence of castration-recurrent prostate cancer in a male patient in need thereof. Methods are described whereby MAGE-11 may serve as a target for vaccine development in the treatment of castration-recurrent prostate cancer. Methods for monitoring endometrial maturation, for diagnosing infertility, and for in vitro fertilization in a female human or nonhuman primate are also provided. Compositions of the invention include antibodies that specifically bind MAGE-11 and oligonucleotide primers useful for detecting MAGE-11 mRNA, as well as kits containing such antibodies or primers.

### FEDERALLY SPONSORED RESEARCH OR DEVELOPMENT

This invention was made with government support under grant numbers 5R01HD16910-23 and 5U54HD035041-09 awarded by the National Institutes of Health/National Institute of Child Health and Human Development and by P01-CA77739 from the National Cancer Institute of the National Institutes of Health. The United States government has certain rights in the invention.

### FIELD OF THE INVENTION

The present invention relates to methods and compositions for the detection of endometrial receptivity to embryo implantation. The present invention also relates to methods and compositions for the detection and treatment of castration-recurrent prostate cancer.

### BACKGROUND OF THE INVENTION

The efficiency of embryo implantation in assisted reproduction procedures is quite low. Typically, such embryo transfers result in pregnancy in only approximately 15% to 25% of patients. In spite of such low efficiency, however, the number and cost of in vitro fertilization (IVF) procedures continue to rise. Approximately 50,000 human IVF procedures are performed in the United States annually and although costs vary widely depending on drugs, testing and other laboratory fees, typical IVF charges are on the order of $10,000 per procedure. Improved knowledge of factors that influence embryo implantation would help reduce embryo loss, help reduce the cost of infertility procedures, and would also help determine the cause for infertility where no other apparent etiology has been identified.

Embryo implantation into the endometrium of the uterus involves a complex sequence of signaling events between the endometrium and embryo, and a large number of molecular mediators involved in this process have been identified to date, including adhesion molecules, cytokines, growth factors, lipids and others. Unfortunately, knowledge of such molecular signals has failed to provide many reliable markers for identifying the window of endometrial receptivity for embryo implantation. A few markers do exist, such as αᵥ/β₃ integrin (see, e.g., U.S. Pat. No. 6,979,533), HOXA10 (see Taylor et al. (1998) J. Clin. Invest. 101:1379-1384), and mouse ascites Golgi factor (MAG, see U.S. Pat. No. 5,599,680). However, these markers have practical limitations to their use. For example, the antibody used in immunohistochemical investigations for the αᵥ/β₃ integrin only works with frozen biopsies and not the more commonly retrieved formalin fixed, paraffin embedded sections. MAG works only in biopsies of blood group A individuals. Expression of HOXA10, as opposed to both αᵥ/β₃ integrin and MAG, cannot be evaluated immunohistochemically and can only be assessed by measuring RNA levels in frozen sections.

The localization of androgen receptors (AR) and their ligands in the uterine microenvironment at early pregnancy suggests a role for ARs in normal uterine physiology (Shiina et al. (2006) Proc. Natl. Acad. Sci. USA 103:224-229). In males, in addition to the function of AR in normal male reproductive development, physiology and health, AR is almost universally expressed in all stages of prostate cancer and overwhelming evidence indicates that AR drives prostate cancer development and progression. Prostate cancer begins as an androgen dependent tumor that responds with remission to surgical or medical castration. However, with time, prostate tumors re-grow despite undetectable circulating androgen levels following androgen deprivation therapy.

The most commonly tested serum marker for the development and progression of prostate cancer is prostate specific antigen (PSA). PSA is normally present in low levels in the blood of all adult men, and these levels are elevated when prostate cancer is present in a manner that correlates with stage and tumor volume. However, a variety of conditions other than prostate cancer can raise PSA levels including prostatitis and benign prostatic hypertrophy, which interferes with the predictive accuracy of PSA as a specific marker for prostate cancer.

Therefore, there is a need in the art for specific and reliable molecular markers for the detection of endometrial receptivity to embryo implantation in women and prostate cancer progression in men.

### SUMMARY OF THE INVENTION

Compositions and methods for determining endometrial receptivity to embryo implantation are provided. The methods involve measuring the protein and messenger RNA (mRNA) levels of melanoma antigen gene protein-11 (MAGE-11, also referred to as MAGE-A11) of the MAGE-A subfamily of MAGE cancer-testis antigens, in an endometrial tissue sample. The level of MAGE-11 protein or the level of expression of MAGE-11 mRNA can be correlated to endometrial receptivity to embryo implantation. Therefore, the invention includes methods to determine the optimum timing window for embryo implantation in a female human or nonhuman primate. The methods may also further include diagnosing infertility and monitoring endometrial maturation by measuring the level of MAGE-11 expression.

Compositions and methods for the detection and treatment of castration-recurrent prostate cancer, are also provided. The methods involve measuring the level of MAGE-11 protein and mRNA in a prostate tissue sample. The level of MAGE-11 protein or the level of expression of MAGE-11 mRNA can be correlated to the presence of castration-recurrent prostate cancer. Therefore, the invention includes methods to diagnose castration-recurrent prostate cancer in a male patient in need thereof. The methods may further include treatment of castration-recurrent prostate cancer in a male patient in need thereof by administering MAGE-11 or a fragment thereof, or an agent that inhibits MAGE-11 function, to the prostate cancer patient.

Compositions of the invention include antibodies that specifically bind MAGE-11 as well as kits containing the antibodies. Compositions further include oligonucleotide primers useful for detecting MAGE-11 RNA and kits containing such primers.

### BRIEF DESCRIPTION OF THE DRAWINGS

FIG. 1 shows the human MAGE-11 amino acid sequence and peptides for antibody production. Shown schematically are human MAGE-11 exons and nuclear localization signal (NLS, underlined). Polyclonal antibodies were raised against MAGE-11 peptides containing amino acid residues 13-26, 59-79 and 94-108 (FIG. 1, shaded gray).

FIG. 2 shows MAGE-11 immunostaining in human endometrium. Shown is immunostaining using 9 μg/ml untreated (A) and peptide antigen preadsorbed antibody MagAb94-108 (B) for mid-secretory LH+6 (patient M187, cycle day 18), 4 μg/ml untreated (C) and preadsorbed MagAb59-79 (D) for mid-secretory LH+6 (patient M124, cycle day 21) and 4 μg/ml untreated (E) and preadsorbed MagAb13-26 (F) for early secretory LH+5 (patient M135, cycle day 15). Brown reaction product represents MAGE-11 immunoreactivity shown against toluidine blue counterstain. Original magnification 40×.

FIG. 3 shows the menstrual cycle stage dependence of MAGE-11, AR, progesterone receptor (PR) and estrogen receptor α (ERα) immunostaining in human endometrium. Paraffin-fixed sections of normal human endometrium were immunostained using antibodies against MAGE-11 (MagAb94-108, 9 μg/ml, A, E, I, M), AR (Abcam Inc., Cambridge, Mass., ab3510, 0.38 μg/ml, B, F, J, N), PR (Santa Cruz Biotechnology, Santa Cruz, Calif., H-190, 2 μg/ml, C, G, K, O) and ERα (NovoCastra, Burlingame, Calif., NCL-ER-6F11, 1:500 dilution, D, H, L, P). Shown are representative sections from proliferative (patient N003, cycle day 9 (CD9), A-D), early secretory LH+5 (patient M135, cycle day 15, E-H), mid-secretory LH+9 (patient M122, cycle day 27, I-L) and late secretory LH+14 (patient M097, cycle day 29, M-P). Brown reaction product represents positive immune reactivity shown against toluidine blue counterstain. Original magnification 60×.

FIG. 4 shows MAGE-11 and AR mRNA levels in normal human endometrial biopsies through the menstrual cycle. (A) Relative MAGE-11 mRNA levels in individual subjects shown by menstrual cycle day or day following the LH surge were assayed using total RNA from frozen human endometrium samples of normal cycling women. Shown are normalized values relative to LH+14. (B) Average relative MAGE-11 mRNA levels by menstrual cycle stage expressed as mean±SD for proliferative phase (cycle days 1-14) and early (LH+1 to LH+5), mid (LH+6 to LH+10) and late secretory stage (LH+11 to LH+14). (C) Relative endometrial AR mRNA levels in individual subjects shown by menstrual cycle day or days following the LH surge. Shown are normalized values of AR mRNA relative to LH+14. (D) Average relative AR mRNA levels by menstrual cycle stage. (E) Schematic of hormone profiles for LH (magenta), 17β-estradiol (blue), progesterone (green), and MAGE-11 mRNA levels (black dotted line) based on the 28 day human menstrual cycle relative to the window of endometrial receptivity to embryo implantation at LH+6 through LH+10 (red box), cycle day and day following the LH surge. Proliferative stage is cycle days 1-13, LH surge at cycle day 14, ovulation at LH+1, early secretory LH+1 to LH+5 (cycle days 15-19), mid-secretory LH+6 to LH+10 (cycle days 20-24) and late secretory LH+11 to LH+14 (cycle days 25-28).

FIG. 5 shows stage dependent changes in MAGE-11, AR and ERα mRNA in normal human endometrium through the menstrual cycle. MAGE-11 (black), AR (gray) and ERα mRNA (white) levels are shown. Relative mean mRNA levels in different stages of the cycle are not indicative of absolute levels but illustrate greater AR than MAGE-11 mRNA levels in the proliferative and late secretory phase and greater MAGE-11 than AR mRNA in the early and mid-secretory stages.

FIG. 6 shows relative levels of MAGE-11 and AR mRNA between different cell lines. (A) MAGE-11 and AR mRNA in cell lines cultured in 10 cm dishes in serum media for 3 days to ˜80% confluency include human cervical carcinoma HeLa (H, 2×10⁶ cells/dish), human endometrial Ishikawa (I, 4×10⁶ cells/dish) and ECC1 (E, 3×10⁶ cells/dish), monkey kidney CV1 (C, 2×10⁶ cells/dish), human prostate cancer LNCaP (L, 8×10⁶ cells/dish) and human normal prostate PWR-1E (P, 4×10⁶ cells/dish) cells. cDNA from 0.4 μg total RNA was assayed by real-time PCR. Serial dilutions with known copy numbers of pCMVhAR, pSG5-HA-MAGE-11, AR DNA fragment coding for amino acid residues 508-660, and MAGE-11 DNA fragment coding for residues 2-179, were amplified to generate standard curves. mRNA copies/μg total RNA were calculated from Ct values using standard curves. (B) The 63 by MAGE-11 123-185 nt (GenBank AY747607.1, upper gel) and 99 by AR 2197-2295 nt (GenBank J03180, lower gel) real-time PCR products were analyzed on 2.5% agarose gels. Abbreviations are as in (A) with pSG5-MAGE-11 or pCMVhAR vector DNA (V) and MAGE-11 and AR PCR fragment DNA (Fg).

FIG. 7 shows estrogen regulation of MAGE-11 and AR mRNA in the human endometrial ECC-1 cell line. ECC-1 cells were cultured and plated in phenol red free, 5% charcoal stripped serum medium and the next day harvested (0 time) or the medium replaced with or without 10 nM E₂ and harvested 24, 48 and 72 h later (A, C). Dose response studies were performed at increasing E₂ concentration or 1 μM ICI-182,780 with and without 0.1 nM E₂ as indicated and harvested 48 h later (B, D). Shown are relative levels of MAGE-11 (A, B) and AR (C, D) mRNA determined from total RNA by real-time PCR from duplicates of two 10 cm dish cultures extrapolated from standard curves and threshold cycle Ct values and expressed as ratios of target gene to control GusB±SD.

FIG. 8 shows cAMP regulation of MAGE-11 and AR mRNA in the human endometrial ECC1 cell line. ECC-1 cells were cultured, plated and treated in phenol red free, 5% charcoal stripped serum medium with and without 2 mM dibutyryl-cAMP (A, C), 2 mM dibutyryl-cAMP with and without 10 nM E₂ (B) and 0.5 mM dibutyryl-cAMP (D) for the times indicated. Shown are relative levels of MAGE-11 (A, B) and AR (C, D) mRNA from duplicates of two 10 cm dish cultures extrapolated from standard curves and threshold cycle Ct values expressed as ratios of target gene to control GusB±SD.

FIG. 9 shows estrogen and cAMP regulation of MAGE-11 and AR mRNA in the human endometrial Ishikawa cell line. Ishikawa cells were cultured, plated and treated in phenol red free, 5% charcoal stripped serum medium with or without 0.1, 0.4 and 2 mM dibutyryl-cAMP and/or 10 nM E₂ for 48 h. Shown are relative MAGE-11 (A) and AR (B) mRNA levels from duplicates of two 10 cm dish cultures extrapolated from standard curves and threshold cycle Ct values expressed as ratios of target gene to control GusB±SD.

FIG. 10 shows immunoblots of PR, ERα, MAGE-11 and AR in human endometrial ECC-1 and Ishikawa cell lines. Proteins extracted from ECC-1 or Ishikawa cells were separated on 10% acrylamide gels containing SDS calibrated using Kaleidoscope prestained molecular weight markers (BioRad, Hercules, Calif.) indicated on the left. (A) For PR, cells were treated with and without 10 nM E₂ for 48 h and protein (50 μg/lane) probed using PR H-190 antibody (Santa Cruz Biotechnology, Santa Cruz, Calif., 1:500 dilution). Human PR-B and PR-A (2 μg/lane) were expressed in COS cells from pSG5hPR-B and pSG5hPR-A as controls. (B) For ERα cells were treated with and without 10 nM E₂ for 24 h and protein (80 μg/lane) probed with mouse monoclonal human ERα antibody (NovoCastra, Burlingame, Calif., NCL-ER-6F11, 1:150 dilution). Human ERα (1 μg protein/lane) was expressed in COS cells from pCMVhERα as control. (C) For MAGE-11, cells were untreated (left) or Ishikawa cells were treated with and without 10 nM E₂ for 48 h and protein (50 μg/lane) probed with MagAb94-108 immunoglobulin G (8 μg/ml, right). Human MAGE-11 (0.5 μg protein/lane) was expressed in COS cells from pSG5-MAGE-11 as control. (D) For AR, cells were treated for 24 h with and without 10 nM DHT and protein (50 μg/lane) probed with AR32 rabbit polyclonal antibody (1 μg/ml). Human AR (2 μg protein/lane) expressed in COS cells from pCMVhAR served as control.

FIG. 11 shows the increase in AR transcriptional activity by MAGE-11 in human endometrial cell lines. ECC-1 (A) and Ishikawa cells (B) were transfected in 12 well plates with 0.1 μg PSA-Enh-Luc, 2 ng pCMVhAR with and without 100 ng pSG5-MAGE-11 using FuGENE 6. In (C), Ishikawa cells were transfected with 25 ng pCMV5 empty vector (p5) or pCMVhAR1-660 (coding for the AR NH₂-terminal through DNA binding domain) with and without 50, 100 or 250 ng pSG5-MAGE-11 as indicated. Luciferase activities expressed as average±S.E. are representative of three independent experiments.

FIG. 12 shows modulation of AR protein levels by MAGE-11 and modulation of MAGE-11 protein levels by AR. (A) Schematic of full-length human AR amino acid residues 1-919 including FXXLF motif ²³FQNLF²⁷, AF1, DNA binding domain (DBD), nuclear localization signal (NLS), ligand binding domain (LBD) and activation function 2 (AF2). (B) Immunoblots of protein extracted from COS cells transfected with 2 μg wild-type pCMVhAR (WT) or pCMVhAR mutants AR-FXXAA with ²³FQNLF²⁷ changed to FQNAA (He et al. (2000) J. Biol. Chem. 275:22986-22994), ARΔAF1 with the Δ142-337 deletion (Zhou et al. (1995) Mol. Endocrinol. 9:208-218), nuclear transport mutant 4KM with R617M, K618M, K632M and K633M mutations (Zhou et al. (1994)J. Biol. Chem. 269:13115-13123) and DNA binding mutant C576A (Zhou et al. (1995) Mol. Endocrinol. 9:208-218) in the absence (upper panel) and presence (lower 2 panels) of 5 μg pCMV-Flag-MAGE-11. Cells were incubated for 24 h in 10% charcoal-stripped serum medium in the absence and presence of 2 and 10 nM DHT as indicated. Cells were extracted and total protein (10 μg/lane) separated in 10% acrylamide gels containing SDS. AR was detected using AR32 rabbit polyclonal antibody (1:100, upper two panels) and Flag-MAGE-11 with anti-Flag M₂ mouse monoclonal antibody (Sigma, St. Louis, Mo., 1:2000, lower panel). A lower portion of the blot probed with mouse monoclonal actin antibody (Abcam Inc., Cambridge, Mass., 1:5000) verified equivalent loading of total protein per lane.

FIG. 13. Increased androgen dependent and independent AR transcriptional activity by MAGE-11 and TIF2. The CWR-R1 human prostate cancer cell line was transfected with and without expression vectors for MAGE-11 and TIF2. PSA-enhancer-luciferase reporter gene activity was assayed after treatment with 100 ng/ml EGF and increasing concentrations of DHT. The data show that coexpression of MAGE-11 and TIF2 increased androgen dependent and independent AR transcriptional activity.

FIG. 14. Increased levels of MAGE-11 mRNA after progression of the CWR22 human prostate cancer xenograft from androgen dependence to androgen independent castration-recurrent growth following androgen deprivation by castration. (A) MAGE-11, (B) AR and (C) TIF2 mRNA levels were determined by real-time RT-PCR of total RNA extracted from CWR22 tumors from intact non-castrated nude mice (0 days) and 2, 6, 12 and 120 days after castration. Recurrent tumors arise after 120 days following castration and represent relapse of the disease. The data show that MAGE-11 mRNA levels increased with CWR22 tumor progression with highest levels coincident with the onset of castration-recurrent disease that arose in the absence of circulating androgen. AR mRNA levels also increased after castration but to a lesser extent than MAGE-11. TIF2 mRNA levels were not predictive of tumor progression.

FIG. 15. Immunostaining of MAGE-11, AR and TIF2 in the CWR22 tumor at different times after castration. CWR22 tumors were extracted from intact non-castrated nude mice and at 2, 6, 12 and 120 days after castration. The recurrent tumor arises after more than 120 days following castration and is characterized by growth in the absence of circulating androgen. Immunostaining was performed on paraffin fixed sections using MAGE-11 antibody MagAb94-108 (8 μg/ml), AR PG21 (Upstate; 1:150 dilution) and TIF2 (BD Transduction Laboratories, 1:300 dilution). Brown reaction product is indicative of positive immune reactivity against a toluidine blue counterstain. Original magnification 40×.

FIG. 16. Log plot of MAGE-11 and AR mRNA levels in clinical specimens of benign prostatic hyperplasia (BPH), androgen dependent and androgen independent castration-recurrent prostate cancer. (A) MAGE-11 and (B) AR mRNA levels were determined from total RNA extracted from tissue samples from patients with benign prostatic hyperplasia (BPH), androgen dependent and castration-recurrent prostate cancer and analyzed by real-time RT-PCR. The data show increased levels of MAGE-11 or AR mRNA were observed in ˜75% of recurrent prostate cancer specimens.

FIG. 17. Regulation of MAGE-11 mRNA by cyclic AMP in LNCaP cells. LNCaP cells were treated with increasing concentrations of dibutyryl-cyclic AMP for 48 h (A), or for increasing times with 7.5 mM dibutyryl-cyclic AMP (B). MAGE-11 mRNA levels were determined by real time RT-PCR. The data show that the MAGE-11 gene was up-regulated by cyclic AMP in the androgen dependent LNCaP prostate cancer cell line.

### DETAILED DESCRIPTION OF THE INVENTION

The present invention provides methods and compositions for detecting endometrial receptivity to embryo implantation, methods for monitoring endometrial maturation, methods for diagnosing infertility, and methods for in vitro fertilization in women and female nonhuman primates. The present invention also provides methods and compositions for the detection and treatment of castration-recurrent prostate cancer. In one embodiment, the methods comprise detecting the level of expression of MAGE-11 in one or more endometrial tissue samples, where the level of MAGE-11 protein or mRNA is correlated with endometrial receptivity. In another embodiment, the methods comprise detecting the level of MAGE-11 in one or more endometrial tissue samples obtained from a plurality of stages of the menstrual cycle of a female human or nonhuman primate. In another embodiment, the methods comprise detecting the level of expression of MAGE-11 in one or more prostate cancer tissue samples, where the level of MAGE-11 protein or mRNA is correlated with the presence of castration-recurrent prostate cancer. In particular embodiments, MAGE-11 is detected at the protein level using antibodies specific to MAGE-11 or at the nucleic acid level using PCR or RT-PCR. Compositions comprise polyclonal and monoclonal antibodies specific to MAGE-11 protein and kits for practicing the methods of the invention. Compositions further comprise nucleic acid sequences useful for detecting RNA for MAGE-11, and kits containing such nucleic acid sequences.

The methods and compositions of the present invention are based upon the discoveries that an AR coregulator identified recently as melanoma antigen gene protein-11 (MAGE-11 or MAGE-A11, Accession No. NP 005357) of the MAGEA family is expressed in a temporal pattern in glandular epithelial nuclei of the human endometrium during the menstrual cycle, and that MAGE-11 expression is elevated in castration-recurrent prostate cancer cells. MAGE-11 is a member of the MAGE gene superfamily of so-called cancer-testis antigens and one of 12 members of the MAGE-A subfamily coded at Xq28 on the human X chromosome (Chomez et al. (2001) Cancer Res. 61:5544-5551; Rogner et al. (1995) Genomics 29:725-731; Simpson et al. (2005) Nat. Rev. Cancer 5:615-625). MAGE-11 shares sequence homology with other members of the MAGE gene family within the highly conserved 3′ coding exon (Bai & Wilson E M (2008) Mol. Cell. Biol., 28, in press). The MAGE-11 gene contains three additional 5′ coding exons unique to MAGE-11 that include a nuclear localization signal (Bai et al. (2005) Mol. Cell. Biol. 25:1238-1257; Irvine & Coetzee (1999) Immunogenetics 49:585).

The temporal pattern of MAGE-11 expression in glandular epithelial nuclei of the human endometrium during the menstrual cycle and its increased expression in castration-recurrent prostate cancer cells makes MAGE-11 useful as a biomarker. A “biomarker” is any gene or protein whose level of expression in a tissue or cell is altered in relation to a physiological condition of interest.

Thus, in one embodiment of the present invention, MAGE-11 is used as a biomarker in which higher levels of MAGE-11 protein or mRNA are correlated with increased endometrial receptivity to embryo implantation or to greater endometrial maturation in female human and non-human primates. The highest levels of MAGE-11 mRNA and protein in endometrial tissue samples coincide with the window of receptivity to embryo implantation.

As used herein, “endometrium” refers to a glandular layer of variable thickness that lines the uterine wall (myometrium) of a female human or nonhuman primate. The endometrium is extremely sensitive to the hormones estrogen and progesterone and is composed of several functional layers. The basalis layer is nearest the myometrium and the functionalis is the layer closer to the surface. This tissue is made of epithelial cells, stromal (or mesenchymal) cells, and endometrial leukocytes. The epithelial cells are either glandular (meaning that they form glands beneath the surface of the endometrium) or luminal (meaning that they line the surface of the endometrium).

In women of reproductive age, the endometrium undergoes cyclical developmental changes based on the ovarian cycle of hormone release. The proliferative stage of endometrial development for women is represented by cycle days 1-13 of an idealized 28 day menstrual cycle. A surge of gonadotropin luteinizing hormone (LH) occurs on day 14, with ovulation occurring on day 15 (LH+1). Secretory phases are: 1) early secretory for cycle days 15-19 (LH+1 to LH+5); 2) mid-secretory for cycle days 20-24 (LH+6 to LH+10); and 3) late secretory for cycle days 25-28 (LH+11 to LH+14). The timing of embryo implantation and corresponding window of endometrial receptivity to embryo implantation is between cycle days 20-24 (LH+6 to LH+10). As described in more detail in the Examples provided below, MAGE-11 is selectively expressed in endometrial cells during the early secretory and mid-secretory phases of the menstrual cycle as compared to the proliferative phase or late secretory phase of the menstrual cycle. Detection of MAGE-11 expression therefore permits the differentiation of endometrial tissue samples taken during the early secretory or mid-secretory phases of the menstrual cycle, and particularly allows for the identification of tissue samples taken during the window of endometrial receptivity to embryo implantation between days 20-24 (LH+6 to LH+10) of the human menstrual cycle.

Therefore, as used herein, the terms “optimum timing window for embryo implantation” or “window of endometrial receptivity” refer to the time period between days 20 (LH+6) to 24 (LH+10) of an idealized 28 day human menstrual cycle. The terms “endometrial receptivity to embryo implantation” or “mature endometrium” refer to the state of the endometrium during the window of endometrial receptivity. Similar cycles are known for other primates and it is within the ordinary skill in the art to adopt methods described herein to such cycles.

Thus, in one embodiment of the present invention, a method for detecting endometrial receptivity to embryo implantation in a female human or nonhuman primate is provided. The method for detecting endometrial receptivity to embryo implantation comprises the steps of: a) obtaining an endometrial tissue sample from the female human or nonhuman primate; b) detecting the level of expression of MAGE-11 in the endometrial tissue sample; and c) correlating the level of expression of MAGE-11 in the endometrial tissue sample with endometrial receptivity to embryo implantation. In some embodiments, the method for detecting endometrial receptivity to embryo implantation comprises detecting the level of expression of MAGE-11 in endometrial tissue samples obtained from a plurality of stages of the menstrual cycle of the female human or nonhuman primate.

In another embodiment of the present invention, a method for monitoring endometrial maturation in a female human or nonhuman primate is also provided. The endometrium may be monitored for embryo receptivity, embryo implantation, infertility, endometrial replenishment and ovulation. The method for monitoring endometrial maturation in a female human or nonhuman primate comprises the steps of: a) obtaining an endometrial tissue sample from a female human or nonhuman primate; b) detecting expression of MAGE-11 in the endometrial tissue sample; c) repeating steps a) and b) with endometrial tissue samples obtained from a plurality of stages of the menstrual cycle of the female human or nonhuman primate; and d) correlating the level of expression of MAGE-11 in one or more tissue samples of step c) with endometrial maturation.

In another embodiment of the present invention, a method of in vitro fertilization in a female human or nonhuman primate is also provided. In one embodiment, the method of in vitro fertilization comprises the steps of: a) obtaining an endometrial tissue sample from the female human or nonhuman primate; b) detecting expression of MAGE-11 in the endometrial tissue sample; c) repeating steps a) and b) with endometrial tissue samples obtained from a plurality of stages of the menstrual cycle of the female human or nonhuman primate; d) correlating the level of expression of MAGE-11 in one or more tissue samples of step c) with endometrial maturation; and e) introducing an embryo into the uterus of the female human or nonhuman primate when the endometrium is mature. In some embodiments, the method of in vitro fertilization further comprises monitoring the embryo for implantation. In further embodiments, the embryo for use within the in vitro fertilization method develops from a zygote formed by the combination of an egg and sperm in vitro.

In another embodiment of the present invention, a method for diagnosing infertility in a female human or nonhuman primate is also provided. In one embodiment, the method for diagnosing infertility comprises the steps of: a) obtaining an endometrial tissue sample from the female human or nonhuman primate; b) detecting expression of MAGE-11 in the endometrial tissue sample; c) repeating steps a) and b) with endometrial tissue samples obtained from a plurality of stages of the menstrual cycle of the female human or nonhuman primate; and d) correlating delayed, reduced, increased, or early expression of MAGE-11 in one or more tissue samples of step c) with infertility in the female human or nonhuman primate.

In another embodiment of the present invention, MAGE-11 is used as a biomarker in which higher levels of MAGE-11 protein or mRNA are correlated with the presence of castration-recurrent prostate cancer. Because prostate cancer cells are initially dependent upon androgens for their growth, androgen ablation therapy (also known as hormonal deprivation therapy) is a well-established form of treatment for various stages of prostate cancer, especially advanced stages of cancer. However, this treatment alone does not cure the disease. During the course of androgen ablation therapy, prostate cancer cells eventually lose their dependency on androgen and become highly aggressive. As used herein, prostate cancer cells are “androgen responsive” if their growth is stimulated by androgens, while “castration-recurrent” (also called “androgen-independent” or “androgen-refractory”) prostate cancer cells do not depend on androgen for their proliferation. Individuals with androgen-independent prostate cancer exhibit a lack of response in prostate specific antigen (PSA) levels in connection with androgen-suppression therapy.

Thus, in one embodiment, a method is provided for detecting castration-recurrent prostate cancer in a male patient in need thereof, said method comprising the steps of: a) obtaining a prostate tissue sample from the male patient; b) detecting the level of expression of MAGE-11 in the prostate tissue sample; and c) correlating the level of expression of MAGE-11 in the prostate tissue sample with the presence of castration-recurrent prostate cancer.

Within the methods of the present invention, the level of MAGE-11 expression can be assessed at the protein or nucleic acid level. Methods for determining the level of expression of MAGE-11 at either the nucleic acid or protein level are well known in the art and include but are not limited to immunoblots (western blots), northern blots, Southern blots, enzyme linked immunosorbent assay (ELISA), immunoprecipitation, immunofluorescence, flow cytometry, immunohistochemistry, nucleic acid hybridization techniques, nucleic acid reverse transcription methods, and nucleic acid amplification methods.

In some embodiments, the level of expression of MAGE-11 within the methods of the present invention is detected on a protein level using, for example, antibodies that are directed specifically against the MAGE-11 protein. The term “antibody” as used herein encompasses monoclonal antibodies, polyclonal antibodies, multispecific antibodies (e.g., bispecific antibodies), and antibody fragments, so long as they exhibit the desired biological activity or specificity. “Antibody fragments” comprise a portion of a full-length antibody, generally the antigen binding or variable region thereof. Interactions between antibodies and a target polypeptide are detected by radiometric, colorimetric, or fluorometric means. Detection of antigen-antibody complexes may be accomplished by addition of a secondary antibody that is coupled to a detectable tag, such as for example, an enzyme, fluorophore, or chromophore. Such antibodies can be used in various methods such as Western immunoblot, ELISA, immunoprecipitation, and immunohistochemistry techniques.

Methods for making antibodies are well known in the art. Polyclonal antibodies can be prepared by immunizing a suitable subject (e.g., rabbit, goat, mouse, or other mammal) with MAGE-11 protein or a fragment thereof as an immunogen. A MAGE-11 protein “fragment,” “portion,” or “segment” is a stretch of amino acid residues of at least about 5, 7, 10, 14, 15, 20, 21 or more amino acids. The antibody titer in the immunized subject can be monitored over time by standard techniques, such as with an enzyme linked immunosorbent assay (ELISA) using immobilized MAGE-11 protein or a fragment thereof. At an appropriate time after immunization, e.g., when the antibody titers are highest, antibody-producing cells can be obtained from the animal, usually a mouse, and can be used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:550-52; Kenneth (1980) in Monoclonal Antibodies: A New Dimension In Biological Analyses (Plenum Publishing Corp., NY); and Lerner (1981) Yale J. Biol. Med., 54:387-402).

Alternative to preparing monoclonal antibody-secreting hybridomas, a monoclonal antibody can be identified and isolated by screening a recombinant combinatorial immunoglobulin library (e.g., an antibody phage display library) with MAGE-11 protein or a fragment thereof to thereby isolate immunoglobulin library members that bind the MAGE-11 protein. Kits for generating and screening phage display libraries are commercially available (e.g., the Pharmacia Recombinant Phage Antibody System, Catalog No. 27-9400-01; and the Stratagene SurfZAP□ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

In some embodiments, the level of expression of MAGE-11 within the methods of the present invention is detected at the nucleic acid level. Nucleic acid-based techniques for assessing expression are well known in the art and include, for example, determining the level of MAGE-11 mRNA in an endometrial or prostate tissue sample. Isolated mRNA can be used in hybridization or amplification assays that include, but are not limited to, Southern or northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding MAGE-11.

In one embodiment, the level of MAGE-11 mRNA in an endometrial or prostate tissue sample involves the process of nucleic acid amplification, e.g., by PCR, followed by the detection of the amplified molecules using techniques well known to those of skill in the art. These detection methods are especially useful for the detection of nucleic acid molecules if such molecules are present in very low numbers. In particular aspects of the invention, MAGE-11 expression is assessed by quantitative RT-PCR (e.g., the TaqMan® System). Such methods typically utilize pairs of oligonucleotide primers that are specific for MAGE-11. Such primers are commercially available. For example, MAGE-11 primers Hs00377815-ml (Applied Biosystems, Foster City, Calif.) amplify a 63 by DNA fragment coding for amino acid residues 24-43 at nucleotides 123-185 unique to MAGE-11 (GenBank AY747607.1) and the probe overlaps the exon 2 and 3 junction. Methods for designing oligonucleotide primers specific for a known sequence are well known in the art.

By “endometrial tissue sample” is intended any sampling of cells, tissues, or fluids in which expression of MAGE-11 in endometrial cells can be detected. Methods for obtaining endometrial tissue samples for analysis include any surgical and non-surgical technique known in the art. Surgical methods include, but are not limited to biopsy, dilation and curettage. Non-surgical methods include, but are not limited to, uterine washings and uterine brushings, with or without immunocytochemical evaluation.

By “prostate tissue sample” is intended any sampling of cells, tissues, or fluids in which expression of MAGE-11 in prostate cancer cells can be detected. Methods for obtaining prostate tissue samples for analysis are well known in the art.

As used herein, the term “primate” includes humans and non-human primates. The term “woman” refers to a human female. The term “man” refers to a human male. In some embodiments, the primate within the methods of the present invention is a human female and the stages of the menstrual cycle are selected from the group consisting of the early secretory phase and the mid-secretory phase. In further embodiments, the primate within the methods of the present invention is a female human and the highest expression levels of MAGE-11 mRNA and protein are detected on days LH+5 to LH+10 which are approximately days 15 to 24 of the menstrual cycle, particularly on days 20 to 24 of the menstrual cycle of the ideal 28 day cycle.

Although the methods of the invention are directed to detecting the level of expression of MAGE-11 in an endometrial or prostate tissue sample, two or more biomarkers may also be used to practice the present invention. It is recognized that detection of more than one biomarker in a tissue sample may be used to detect endometrial receptivity to embryo implantation or to detect a mature endometrium or to detect the presence of castration-recurrent prostate cancer within the methods of the invention. Therefore, in some embodiments, two or more biomarkers are used, more preferably, two or more complementary biomarkers. By “complementary” is intended that detection of the combination of biomarkers in a tissue sample results in the successful identification of endometrial receptivity to embryo implantation or of a mature endometrium or of the presence of castration-recurrent prostate cancer in a greater percentage of cases than would be identified if only one of the biomarkers was used. Additional biomarkers for the detection of endometrial receptivity to embryo implantation or the detection of a mature endometrium include, but are not limited to, the β₃ subunit of αᵥ/β₃ integrin (see, e.g., U.S. Pat. Nos. 6,979,533; 6,960,445; 6,733,979; 5,854,401; 5,578,306; 5,478,725; and 5,279,941), the mouse ascites Golgi factor (MAG, see U.S. Pat. No. 5,599,680), and the PUP-1 glycoprotein (see, e.g., U.S. Pat. No. 6,309,843), the disclosures of which are incorporated herein by reference in their entireties. Additional biomarkers for the detection of castration-recurrent prostate cancer include, but are not limited to, p27 (see, e.g., U.S. Pat. No. 6,972,170), prostate specific antigen (PSA; see, e.g., U.S. Pat. No. 5,672,480), and the human androgen receptor (see, e.g., U.S. Pat. Nos. 6,307,030; 6,821,767; and 7,129,078), the disclosures of which are incorporated herein by reference in their entireties.

The present invention also relates to compositions comprising monoclonal or polyclonal antibodies that specifically bind to MAGE-11 protein. As described in more detail in the Experimental section below, polyclonal antibodies MagAb94-108, MagAb59-79 and MagAb13-26 were raised against human MAGE-11-94-108 ⁹⁴ITQIFPTVRPADLTR¹⁰⁸ (SEQ ID NO:1), MAGE-11-59-79 ⁵⁹DLPRVQVFREQANLEDRSPRR⁷⁹ (SEQ ID NO:2) and MAGE-11-13-26 ¹³SPASIKRKKKREDS²⁶ (SEQ ID NO:3) peptides, respectively, containing in addition an NH₂-terminal cysteine linker (Pocono Rabbit Farm & Laboratory, Inc., Canadensis, Pa.). Thus, the present invention also relates to the polyclonal antibodies MagAb94-108, MagAb59-79 and MagAb13-26 and, in particular embodiments of the methods of the present invention, polyclonal antibodies MagAb94-108, MagAb59-79 and MagAb13-26 are used to detect MAGE-11 protein levels. Two additional rabbit polyclonal antibodies that recognize the human MAGE-11 protein by immunoblotting, immunoprecipitation and immunohistochemistry were raised against the full-length MAGE-11, which contains an NH₂-terminal FLAG tag (Flag-MagAb).

The present invention also relates to kits for practicing the methods of the invention. By “kit” is intended any article of manufacture (e.g., a package or a container) comprising at least one antibody directed to MAGE-11 and chemicals for the detection of antibody binding to MAGE-11, or at least one pair of oligonucleotide primers specific for MAGE-11. The kit may be promoted, distributed, or sold as a unit for performing the methods of the present invention. Additionally, the kits may contain a package insert describing the kit and instructions for using the antibody directed to MAGE-11 or the oligonucleotide primers specific for MAGE-11 within the methods of the present invention. In one embodiment, the instructions describe methods for detecting endometrial receptivity to embryo implantation, monitoring endometrial maturation, diagnosing infertility, or for in vitro fertilization in a female human or nonhuman primate. In another embodiment, the instructions describe methods for treating castration-recurrent prostate cancer in a male patient in need thereof. In a particular embodiment, the kit of the invention comprises the antibody MagAb94-108, MagAb59-79, MagAb13-26, or Flag-MagAb and instructions for use of the antibody within the methods of the present invention.

The present invention also relates to methods for treating castration-recurrent prostate cancer in a male patient in need thereof. In one embodiment, a human MAGE-11 protein or fragment thereof may be used as a vaccine for treating castration-recurrent prostate cancer in a male patient in need thereof. MAGE-11 is a cancer-testis antigen displayed on the cell surface in association with the integral membrane class I major histocompatibiilty complex (MHC) and is recognized by T-cell receptors, which leads to destruction by killer T cells. Cancer testis antigens are targets for vaccine immunotherapy because their presentation on the cell surface by the class I MHC complex elicits a T cell immune response (Simpson et al. (2005) Nat. Rev. Cancer 5:615-625).

Thus, in one embodiment, a method is provided for stimulating an immune response in a male patient in need thereof comprising administering a human MAGE-11 protein or fragment thereof to the patient. In another embodiment, a method is provided for treating castration-recurrent prostate cancer in a male patient in need thereof comprising administering a human MAGE-11 protein or fragment thereof to the patient. In particular embodiments, the MAGE-11 fragment for use within these methods comprises the amino acid sequence set forth in SEQ ID NO:1, SEQ ID NO:2, or SEQ ID NO:3.

In another embodiment, the invention relates to methods for treating castration-recurrent prostate cancer in a male patient in need thereof using an agent that inhibits MAGE-11 function. Agents that inhibit MAGE-11 function include, for example, siRNA, miRNA, antisense RNA, and antisense DNA that interfere with MAGE-11 gene expression, or antagonists of the MAGE-11 protein, such as anti-MAGE-11 antibodies as described elsewhere herein.

Thus, in one embodiment, a method is provided for inhibiting the growth of castration-recurrent prostate cancer cells in a male patient in need thereof comprising contacting the cells with an agent that inhibits MAGE-11 function. In another embodiment, a method is provided for treating castration-recurrent prostate cancer in a male patient in need thereof comprising administering an agent that inhibits MAGE-11 function to said patient. In particular embodiments, the agent that inhibits MAGE-11 function is an siRNA, an miRNA, an antisense RNA, an antisense DNA, or an antagonist of the MAGE-11 protein. In one embodiment, the antagonist of the MAGE-11 protein is an antibody that specifically binds to human MAGE-11 protein or fragment thereof. In another embodiment, the antagonist of the MAGE-11 protein is an antibody that specifically binds to a MAGE-11 fragment comprising the amino acid sequence set forth in SEQ ID NO:1, SEQ ID NO:2, or SEQ ID NO:3. In another embodiment, the antagonist of the MAGE-11 protein is the antibody MagAb94-108, MagAb59-79, MagAb13-26 or Flag-MagAb.

The following examples are offered by way of illustration and not by way of limitation.

### EXPERIMENTAL

### Example 1

### Cycle-Dependent Expression of the Androgen Receptor Coregulator MAGE-11 in Human Endometrium

The requirement for androgen receptor (AR) mediated gene regulation in male sex development is clearly demonstrated by individuals with the androgen insensitivity syndrome. In this X chromosome linked disorder, 46XY genetic males with single missense mutations in the AR gene are born with ambiguous genitalia or complete female external genitalia. Identical loss-of-function mutations have relatively little phenotypic effect in 46XX carrier females (Quigley et al. (1995) Endocr. Rev. 16:271-321) in part due to the double allele status of the AR gene and to random and partial inactivation of the X chromosome. However, female mice homozygous for AR inactivating mutations have reproductive abnormalities that implicate a role for AR in female fertility (Shiina et al. (2006) Proc. Natl. Acad. Sci. USA 103:224-229). Reduced fertility in homozygous female AR knockout mice is associated with prolonged estrous cycles, fewer oocytes after superovulation, follicular atresia and diminished endometrial growth (Hu et al. (2004) Proc. Natl. Acad. Sci. USA 101:11209-11214). Mechanisms of AR action in normal female physiology remain poorly understood.

AR transcriptional activity depends on activation function 1 (AF1) in the NH₂-terminal region and activation function 2 (AF2) in the ligand binding domain, both of which serve as interaction sites for coregulator proteins that bridge to the transcriptional machinery (Heinlein and Chang (2002) Endocr. Rev. 23:175-200). One AR coregulator identified recently is the X chromosome linked melanoma antigen gene protein-11 (MAGE-11, also known as MAGE-A11) of the MAGEA gene family. MAGE-11 was identified as an AR interacting protein in a yeast two hybrid screen of a human testis library using the AR NH₂-terminal FXXLF motif as bait (Bai et al. (2005) Mol. Cell. Biol. 25:1238-1257). MAGE-11 is one of the so-called cancer-testis antigens and is expressed in primates but not in rats, mice or other mammals. MAGE-11 binds the AR FXXLF motif, stabilizes the ligand-free AR and increases androgen dependent AR transcriptional activity (Bai et al. (2005) Mol. Cell. Biol. 25:1238-1257). Binding of MAGE-11 to the AR FXXLF motif inhibits the androgen dependent AR NH₂- and carboxyl-terminal (N/C) interaction between the AR FXXLF motif and AF2. The transcriptional activity of AF2 is determined by competitive binding of the AR FXXLF motif, SRC/p160 coactivator LXXLL motifs and FXXLF motifs in putative AR coregulators (He et al. (2002) J. Biol. Chem. 277:10226-10235; Hsu et al. (2003) J. Biol. Chem. 278:23691-23698).

AR is required for normal female reproductive function (Shiina et al. (2006) Proc. Natl. Acad. Sci. USA 103:224-229) and MAGE-11 expression in tissues and cell lines from the human female reproductive tract correlates with AR expression (Bai et al. (2005) Mol. Cell. Biol. 25:1238-1257). Based on its ability to increase AR transcriptional activity by stabilizing AR and facilitating SRC/p160 coactivator recruitment, the following study describes experiments to determine whether MAGE-11 provides a signal amplification mechanism to compensate for low circulating testosterone levels in the female. The results showed that MAGE-11 was expressed in a striking temporal fashion in human endometrium during the menstrual cycle. Highest levels of MAGE-11 coincided with the window of uterine receptivity to embryo implantation. MAGE-11 expression was tightly controlled in human endometrial cell lines by steroids and second messengers consistent with its endometrial expression profile and the dynamic hormone flux of the menstrual cycle.

### Methods

MAGE-11 antibodies. Rabbit polyclonal antibodies were raised against human MAGE-11 NH₂-teminal peptides ⁹⁴ITQIFPTVRPADLTR¹⁰⁸, ⁵⁹DLPRVQVFREQANLEDRSPRR⁷⁹ and ¹³SPASIKRKKKREDS²⁶ containing in addition an NH₂-terminal cysteine linker (Pocono Rabbit Farm & Laboratory, Inc., Canadensis, Pa.). Antibodies were purified by peptide affinity chromatography using Affi-Gel 10 (Bio-Rad, Hercules, Calif.) coupled to antigen in 0.2 M ethanolamine, pH 8.0, eluted using 0.1 M glycine, pH 3.0, neutralized with 0.1 volume of 1 M Tris-HCl, pH 8.0 (Bai et al. (2005) Mol. Cell. Biol. 25:1238-1257), amended to 0.05 M NaCl and 5% glycerol and stored at −80° C. Antibody specificity was verified by preadsorption and immunoblot analysis of human MAGE-11 (pSG5-MAGE-11) expressed in COS cells which migrates on SDS polyacrylamide gels as 67±3 kDa depending on the molecular weight marker calibration.

Endometrial tissue sampling. Endometrial biopsies were obtained under approved IRB protocols with informed consent at different stages of the menstrual cycle from 21 healthy cycling women volunteers 18-35 years of age with 25-35 day intermenstrual intervals. Women were excluded who used hormone contraception or medications that alter reproductive hormone levels or had infertility or upper reproductive tract disease. Cycle day was determined by the first day of menstruation. Urinary LH was determined by a home test kit (Ovuquick One Step, Conception Technologies, San Diego Calif.). Endometrial biopsies were obtained from proliferative, early, middle and late secretory stages and excluded for evidence of inflammation, hyperplasia or neoplasia. Study participants were randomized for endometrial sampling on a specific predetermined day after the onset of menstruation for proliferative phase samples and a specific day after the urinary LH surge for post-ovulatory samples. Endometrium samples were classified by patient reported cycle day and number of days after the LH surge. Histological staging of hematoxylin and eosin stained fixed sections according to Noyes et al. (Noyes et al. (1975) Am. J. Obstet. Gynecol. 122:262-263) served to confirm cycle stage but no changes were made to cycle day or phase based on histological criteria. Samples for immunostaining were placed in 10% neutral buffered formalin in the clinic within 10 min of surgical removal. Samples for RNA extraction were flash frozen and stored at −80° C.

Immunostaining. Paraffin embedded serial sections (8 μm) of normal human endometrium were immunostained using deparaffinized fixed sections treated with 83% methanol and 5% H₂O₂ at room temperature to reduce endogenous peroxidase activity.

Tissue sections were not treated further for rabbit polyclonal MAGE-11 antibody MagAb94-108 (9 μg/ml) and PR H-190 antibody (Santa Cruz Biotechnologies, Santa Cruz, Calif., sc-7208, 2 μg/ml). For MAGE-11 antibodies MagAb59-79 (4 μg/ml) and MagAb13-26 (4 μg/ml), sections were further treated with 0.05 mg/ml trypsin for 5 min at room temperature followed by washing in cold PBS. For preadsorption studies, antibodies were preincubated with 0.1 or 0.2 mg/ml for 2 days at 4° C. with respective peptide antigens, centrifuged for 10 min at 4° C. at 12,600 xg and used under identical conditions as untreated antibody. For rabbit polyclonal AR antibody (Abeam Inc., Cambridge, Mass., ab3510, 0.38 μg/ml) and mouse monoclonal human ERα antibody (NovoCastra, Burlingame, Calif., NCL-ER-6F11, 1:500 dilution), tissue sections were exposed to 0.01 M sodium citrate, pH 6.0 for 15 min in a microwave at high setting (Balaton et al. (1993) Ann. Pathol. 13:188-189). Sections were blocked with 2% normal goat serum, incubated overnight at 4° C. in a humidified chamber with primary antibody and blocked again with 2% normal goat serum followed by a 1 h incubation at room temperature with biotinylated secondary antibody (Vector Labs, Burlingame, Calif.). Slides were incubated with avidin DH-biotinylated horseradish peroxidase H complex (Vectastain Standard ABC kit, Vector Labs, Burlingame, Calif.) for 1 h at room temperature followed by immersion in 3,3′-diaminobenzidine tetrahydrochloride (Aldrich Chemical Co., Milwaukee, Wis.) at 150 mg/200 ml 0.05 M Tris-HCl buffer containing 0.002% hydrogen peroxide for 10 min with constant stirring. Sections were exposed to osmium vapors and counterstained with 0.05% toluidine blue in 30% ethanol, dehydrated, cleared in xylene and mounted with Permount (Fisher, Pittsburgh, Pa.). Photographs were taken using a SPOT-4 Megapixel Digital Camera (Diagnostic Instruments, Inc., Sterling Heights, Mich.) attached to a Nikon ECLIPSE E600 microscope and prepared using SPOT image processing software.

Real-time PCR. MAGE-11 and AR mRNA were measured using total RNA extracted from frozen endometrial biopsy tissue with RNAqueous-4 PCR kit (Ambion, Austin, Tex.). First strand cDNA was synthesized using SuperScript II reverse transcriptase (Invitrogen, Carlsbad, Calif.). Real-time reverse transcriptase PCR quantitation was performed using Taqman chemistry and the delta delta Ct relative quantitation method using peptidylprolyl isomerase A (cyclophilin A) constitutive housekeeping control. Endometrial biopsy derived cDNA analysis was performed on a Stratagene Mx3000 (Stratagene, La Jolla, Calif.). The peptidylprolyl isomerase A TaqMan Gene Expression Assay Mix Hs99999904-ml (Applied Biosystems, Foster City, Calif.) amplifies a 98 bp 317-414 nucleotide (nt) DNA fragment coding for amino acid residues 102-133 in exon 4 (GenBank Y0052). MAGE-11 TaqMan Mix Hs00377815-ml (Applied Biosystems, Foster City, Calif.) amplifies a 63 by 123-185 nt DNA fragment coding for amino acid residues 24-43 with the probe centered at 154 nt (GenBank AY747607.1) overlapping the exon 2 and 3 junction. AR TaqMan Mix Hs00907244-ml (Applied Biosystems, Foster City, Calif.) amplifies a 99 by 2197-2295 nt DNA fragment coding for AR amino acid residues 612-644 with the probe centered at 2244 nt (Lubahn et al. (1988) Mol. Endocrinol. 2:1265-1275) overlapping the exon 3 and 4 junction. ERα TaqMan Mix Hs174860-ml (Applied Biosystems, Foster City, Calif.) amplifies a 62 by 1093-1154 nt DNA fragment corresponding to amino acid residues 245-264 at assay location 1124 nt (GenBank NM-000125) spanning the exon 3 and 4 boundary. Samples were analyzed in triplicate and efficiency of primer probe sets confirmed for each run using serial dilutions of a standardized sample.

For MAGE-11 and AR mRNA in the human endometrial ECC-1 and Ishikawa cell lines, cells were passed 4 days prior to each experiment into medium without phenol red containing 5% charcoal stripped serum (Atlanta Biologicals, Lawrenceville, Ga.). One day after plating, ECC-1 (2.5×10⁶/10 cm dish or 1×10⁶/6 cm dish) and Ishikawa cells (5×10⁶/10 cm dish) were treated in phenol red free, 5% charcoal stripped serum medium for the indicated times with 0.01-10 nM 17β-estradiol (Sigma, St. Louis, Mo.), 0.1-2 mM dibutyryl-cAMP (Biomol International, Plymouth Meeting, Pa.), 100 Units/ml hCG (Sigma, St. Louis, Mo.), 1 μM ICI-182,780 (Sigma, St. Louis, Mo.), 50 μM forskolin, 10 nM DHT, 10 nM progesterone or 10 ng/ml EGF. RNA was extracted using RNeasy Plus Mini kit (Qiagen, Valencia, Calif.) and cDNA prepared from 4 μg total RNA as above. β-Glucuronidase (GusB) forward primer 5′-TGGTGCTGAGGATTGGCA-3′ (SEQ ID NO:4) and reverse primer 5′-TAGCGTGTCGACCCCATTC-3′ (SEQ ID NO:5) amplify a 65 by region coding for amino acid residues 120-140. The 5′-TGCCCATTCCTATGCCATCGTGTG-3′ (SEQ ID NO:6) GusB probe overlaps the exon 2 and 3 boundary. PCR was carried out in 20 μl reactions containing cDNA from 0.4 μg total RNA, 4 μl LightCycler TaqMan Master mix (Roche, Indianapolis, Ind.) and 0.5 μl 20× TaqMan Mix (Applied Biosystems, Foster City, Calif.) for AR or MAGE-11, or 0.5 μM primer and 0.2 μM probe for GusB. PCR reactions were 1 cycle at 95° C. for 10 min followed by 55 cycles of 95° C. for 15 sec, 60° C. for 25 sec and 72° C. for 1 sec in a Roche Lightcycler. Four serial 10-fold dilutions of cDNA or plasmid DNA were amplified in duplicate to construct standard curves using LightCycler software. mRNA levels were extrapolated based on standard curve and Ct values and normalized to GusB, which except for DHT, was constant under the test conditions and expressed as ratios of target gene to GusB. Results were averaged from duplicates of 6 or 10 cm dishes of ECC-1 or Ishikawa cell cultures.

Transcription assays. Human endometrial ECC-1 and Ishikawa cells (7.5×10⁴/well of 12 well plates) were transfected using FuGENE-6 (Roche Applied Science, Indianapolis, Ind.) with 0.1 μg PSA-Enh-Luc reporter vector, 2 ng pCMVhAR or 25 ng pCMVhAR1-660 and 50-250 ng pSG5-MAGE-11. After 24 h, cells were placed in serum free medium with or without 1 nM DHT and assayed the next day for luciferase activity using a Lumistar Galaxy (BMG Labtech, Durham, N.C.) multiwell plate luminometer.

Immunoblot analysis. MAGE-11, AR, PR and ERα protein levels in ECC-1 (2×10⁶/6 cm dish) and Ishikawa cells (4×10⁶/10 cm dish) were determined by immunoblot. Cells were plated in medium without phenol red containing 5% charcoal stripped serum after culturing in the same media for 4 days. Cells were treated, washed and harvested in cold phosphate buffered saline containing 1 mM dithiothreitol, 1 mM phenylmethylsulfonyl fluoride, 5 μg/ml leupeptin, 5 μg/ml pepstatin A and 5 μg/ml aprotinin, and solubilized in buffer containing 1% Triton X-100, 0.15 M NaCl, 0.5 mM EDTA, 1% sodium deoxycholate, 0.1% SDS, 50 mM Tris-HCl, pH 7.4 and protease inhibitors listed above. AR and MAGE-11 protein interactions were performed in COS cells (1.8×10⁶ cells/10 cm dish) transfected using DEAE dextran with 2 μg wild-type or mutant pCMVhAR and 5 μg pCMV-FLAG-MAGE-11. Cells were washed, harvested in cold phosphate buffered saline and solubilized as above. Protein concentration was determined by BioRad assay using bovine serum albumin as standard. Extracts were separated on 10% acrylamide gels containing SDS and probed with MagAb94-108 immunoglobulin G (8 μg/ml at 4° C.), and rabbit polyclonal AR32 (1 μg/ml), PR H-190 (Santa Cruz Biotechnology, Santa Cruz, Calif., 1:500 dilution) or mouse monoclonal human ERα antibody (NovoCastra, Burlingame, Calif., NCL-ER-6F11, 1:150 dilution). COS cell expression of pCMVhAR, pSG5-MAGE-11, pSG5-PR-B, pSG5-PR-A and pCMVhERα served as positive controls.

### Results

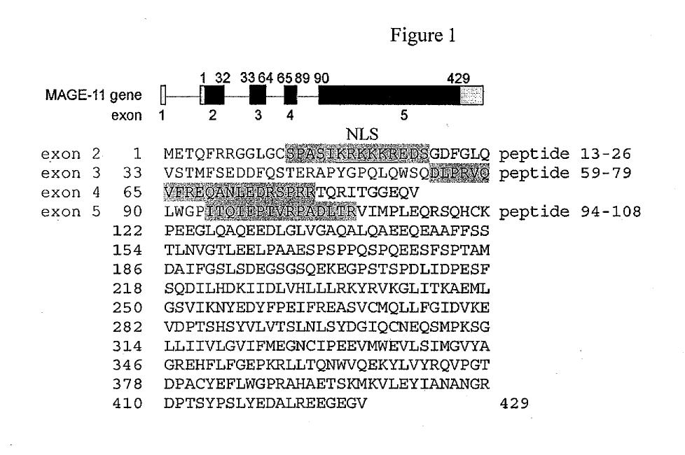

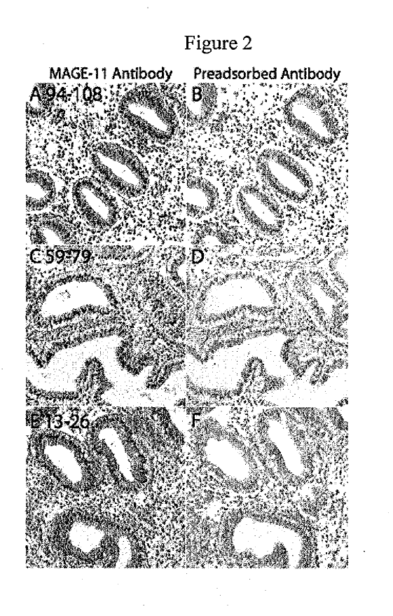

Menstrual cycle dependent MAGE-11 expression in human endometrium. Antibodies were raised against MAGE-11 peptides 13-26, 59-79 and 94-108 coded by separate exons of the MAGE-11 gene (FIG. 1). Each antibody produced strong immunoreactivity in glandular epithelial and stromal cell nuclei of normal human endometrium 5 and 6 days after the midcycle luteinizing hormone (LH) surge (FIG. 2) that was eliminated by preadsorbing the antibodies with the respective peptide immunogens.

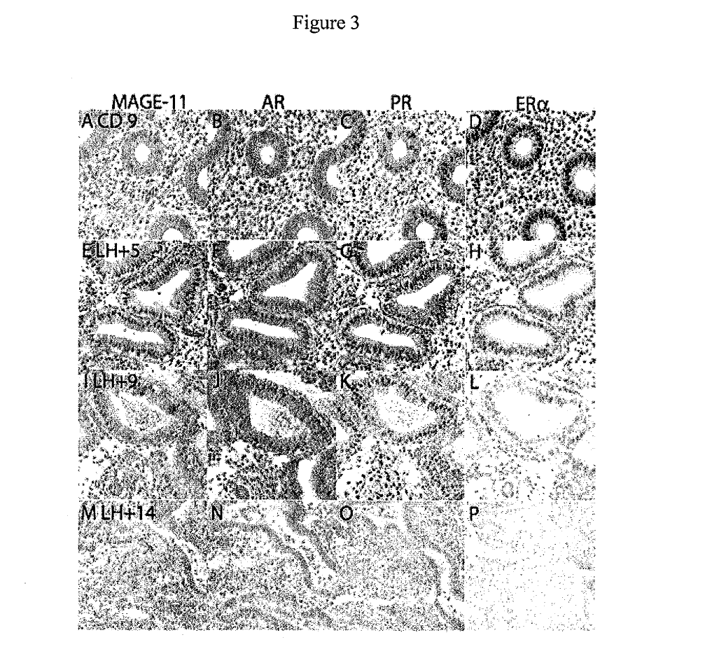

Timing of MAGE-11 expression was investigated in serial sections of endometrial biopsies obtained from normally cycling women during the proliferative phase and early, mid and late secretory stages of the menstrual cycle. Tissue sections were immunostained using MagAb94-108 antibody and antibodies specific for AR, the progesterone receptor (PR) and estrogen receptor-α (ERα). The majority of proliferative stage endometrial specimens lacked prominent immunostaining for MAGE-11 in the small symmetrical glands that characterize this phase of the cycle, with somewhat greater immunoreactivity in stromal cell nuclei (FIG. 3A). Immunostaining of AR and PR was similarly weak in glandular epithelial nuclei throughout the proliferative stage but more prominent in stromal cell nuclei (FIGS. 3B and 3C). In contrast, ERα immunostaining was intense in glandular epithelial and stromal cell nuclei throughout the proliferative phase (FIG. 3D).

Early to mid-secretory stage endometrium is characterized by elongating glands and prominent subnuclear vacuoles (Noyes et al. (1975) Am. J. Obstet. Gynecol. 122:262-263; Murray et al. (2004) Fertil. Steril. 81:1333-1343). Strong MAGE-11 immunostaining was found in glandular epithelial and stromal cell nuclei in the early secretory stage LH+5 that paralleled increasing AR in epithelial cells (FIGS. 3E and 3F). Immunostaining for PR was evident in the early secretory stage and declined for ERα (FIGS. 3G and 3H). MAGE-11 and AR persisted in the mid-secretory stage LH+9, and ERα and PR were heterogeneous between the glands (FIGS. 3I to 3L). By the late secretory stage LH+14, MAGE-11, AR, PR and ERα were nearly undetectable (FIGS. 3M to 3P).

The results demonstrated parallel expression profiles for MAGE-11 and AR in glandular epithelial nuclei during the early and mid-secretory stage of human endometrium when ERαlevels decline.

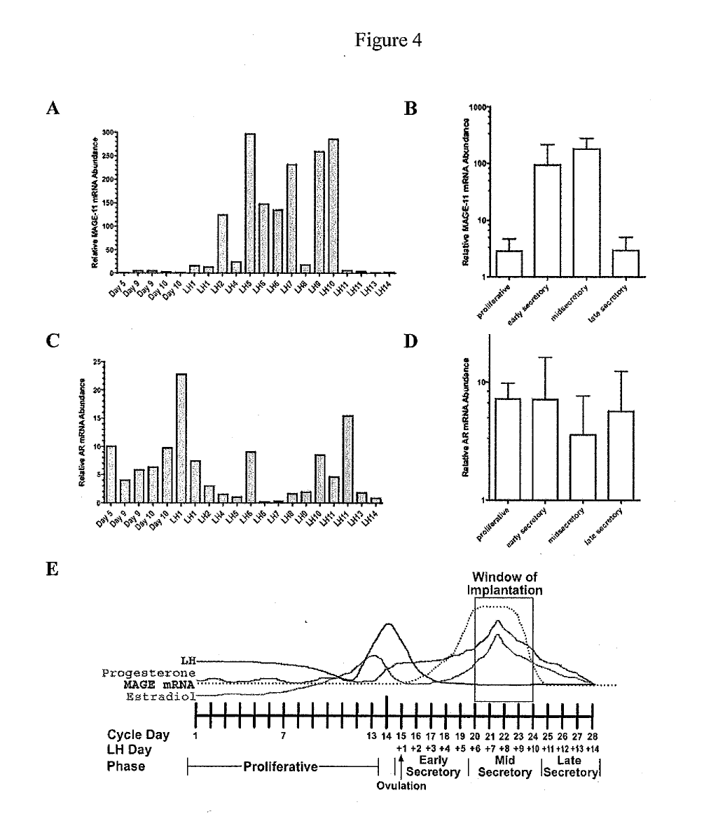

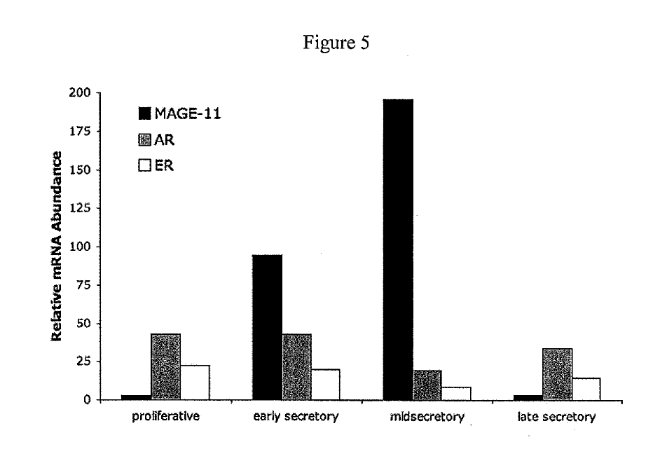

MAGE-11 mRNA expression in normal cycling human endometrium. To investigate further the timing of MAGE-11 expression, RNA was extracted from frozen endometrial biopsies of normally cycling women at different stages in the cycle. It was found that endometrial MAGE-11 mRNA levels measured by real-time PCR paralleled our immunostaining results. MAGE-11 mRNA was low during the proliferative phase shown for menstrual cycle days 5 through 10 (FIG. 4A), increased slightly at LH+1, more significantly by LH+2, was elevated between LH+5 and LH+10, and declined sharply at LH+11. There was a 30 to 70 fold increase in MAGE-11 mRNA during the early and mid-secretory period relative to the proliferative and late secretory stage (FIG. 4B) and timing of maximal MAGE-11 expression coincided with the window of receptivity to embryo implantation at LH+6 through LH+10 (FIG. 4E). AR mRNA levels were highest during the proliferative and late secretory stage and declined in the early and mid-secretory period (FIG. 4C) with no significant difference when grouped by cycle stage (FIG. 4D). The relative increase in MAGE-11 mRNA during the early and mid-secretory period contrasted declining AR and ERα mRNA levels (FIG. 5).

The data support menstrual cycle dependent expression of MAGE-11 in human endometrium coincident with the window of receptivity to embryo implantation. A cycle dependence of AR is more complex since AR mRNA levels declined in the mid-secretory stage when AR and MAGE-11 immunostaining was strong, suggesting MAGE-11 may stabilize AR in the mid-secretory endometrium.

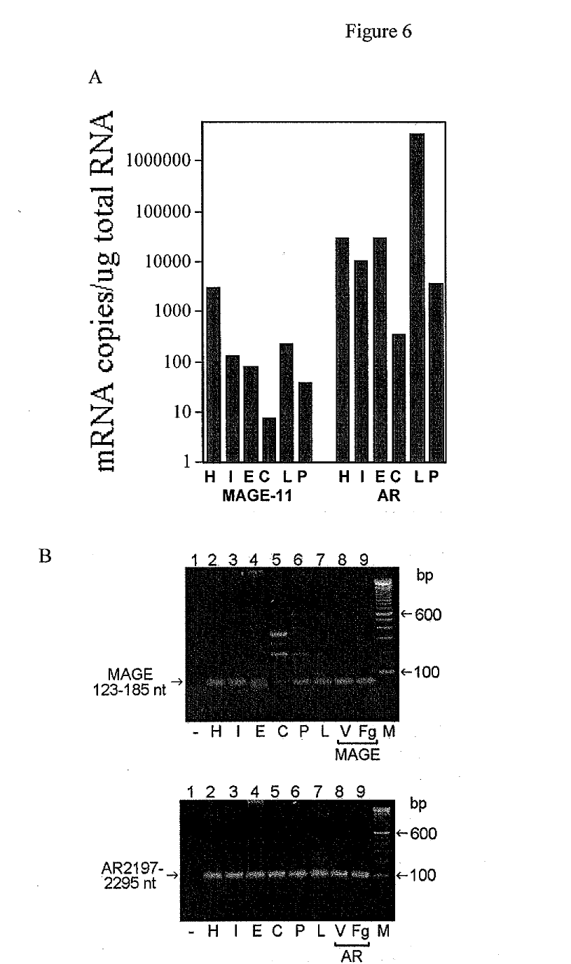

Data from endometrial biopsies indicate AR mRNA levels exceed MAGE-11 by 2-20 fold during the proliferative and late secretory stage, whereas MAGE-11 mRNA levels exceed AR by 10 to 100 fold during the early and mid-secretory phase (FIG. 5). When PCR efficiencies were normalized and specificity of PCR primers and probes established (FIG. 6B), MAGE-11 mRNA levels were ˜100 fold lower than AR mRNA in untreated ECC-1 and Ishikawa human endometrial carcinoma cell lines (FIG. 6A). MAGE-11 mRNA represents ˜100 copies/μg total RNA in the endometrial cell lines, which was ˜10 fold less than the human cervical carcinoma HeLa cell line but similar to MAGE-11 mRNA levels in the LNCaP human prostate cancer cell line. AR mRNA levels in both endometrial cell lines were ˜5 times higher than a normal human prostate cell line (PWR-1E) and ˜100 fold less than LNCaP prostate cancer cells (FIG. 6A). Relative amounts of MAGE-11 mRNA between the cell lines tended to parallel AR mRNA even though MAGE-11 mRNA was low compared to AR.

Endometrial dating. The original report of Noyes et al. (Noyes et al. (1975) Am. J. Obstet. Gynecol. 122:262-263) provided histological classification of the developing human endometrium during the secretory (luteal) phase. Histological landmarks included epithelial mitoses, nuclear pseudostratification and subnuclear vacuoles. The staging scheme of Noyes et al. placed subnuclear vacuoles between LH+2 and LH+4. However, in agreement with a recent report of normally cycling women (Murray et al. (2004) Fertil. Steril. 81:1333-1343), the present results showed persistence of subnuclear vacuoles into endometrial stage LH+5 and LH+6 and later in the mid-secretory period. With these latter criteria, a more consistent temporal alignment was observed between MAGE-11 mRNA expression and the window of receptivity to embryo implantation suggesting MAGE-11 can serve as a biomarker for endometrial staging.

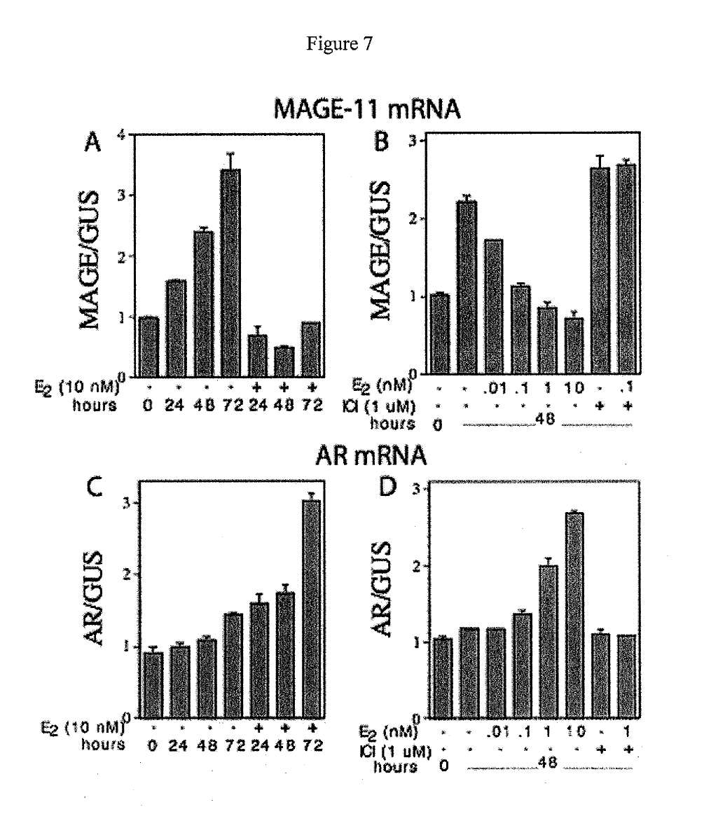

Hormone regulation of MAGE-11. Hormone regulation of MAGE-11 was investigated in the well-differentiated ECC-1 and Ishikawa human endometrial cell lines that express AR, ERα and PR (Tabibzadeh et al. (1990) In Vitro Cell Dev. Biol. 26:1173-1179; Hanifi-Moghaddam et al. (2005) J. Clin. Endocrinol. Metab. 90:973-983; Mo et al. (2006) Biol. Reprod. 75:387-394; Nishida et al. (1996) Hum. Cell 9:109-116; Lovely et al. (2000) J. Steroid Biochem. Mol. Biol. 74:235-241). An increase in MAGE-11 mRNA levels in ECC-1 cells was observed during 72 h of culture in phenol red-free, charcoal stripped serum medium in the absence of added hormone which was blocked by 10 nM estradiol (E₂) in a dose dependent manner, with partial inhibition at 0.01 nM E₂ (FIGS. 7A and 7B). The inhibitory effect of E₂ required more than 6 h and appeared to be ERα mediated since the ICI-182,780 antagonist increased MAGE-11 mRNA levels in the absence and presence of E₂ (FIG. 7B). The increase in MAGE-11 mRNA over time appeared to reflect a temporal loss of estrogenic activity in charcoal stripped serum. This was supported by a ˜7 fold increase in MAGE-11 mRNA in serum free cultures. In contrast, AR mRNA levels increased ˜3 fold in response to 10 nM E₂ (FIG. 7C), requiring 1 nM E₂ which was blocked by ICI-182,780 (FIG. 7D).

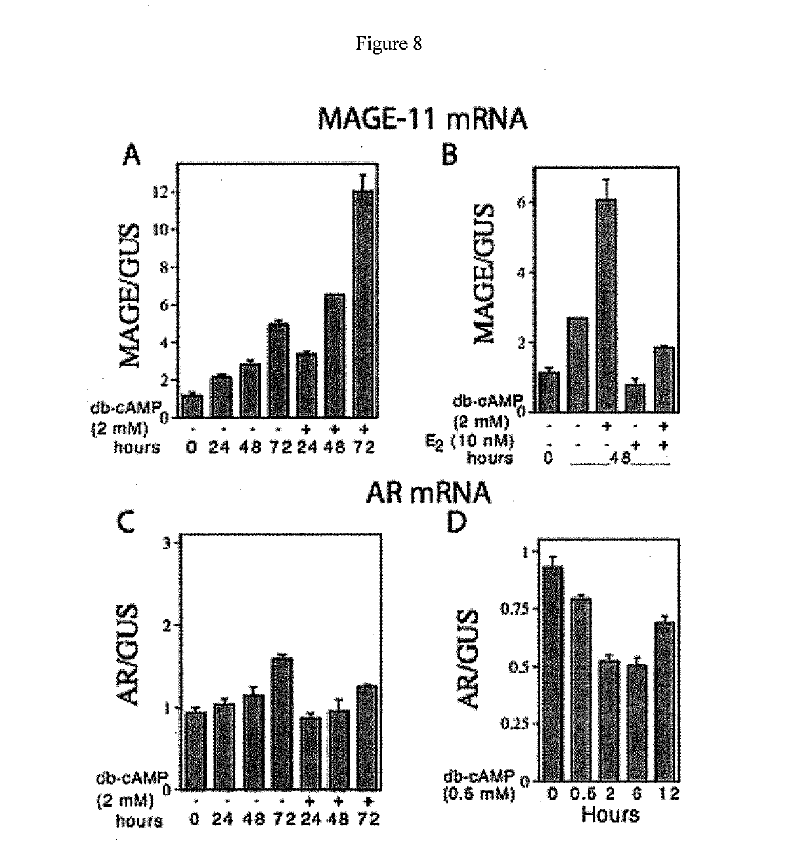

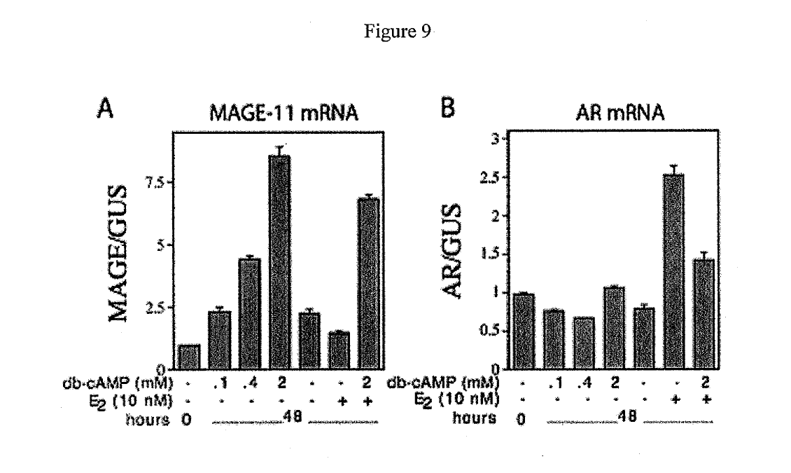

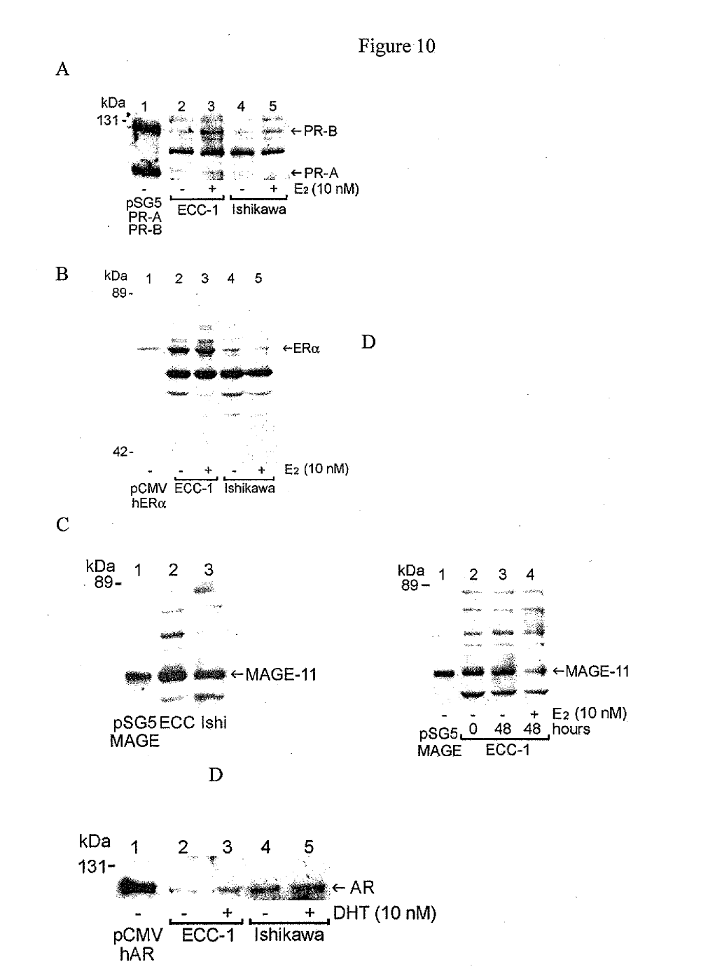

MAGE-11 mRNA levels increased 8-12 fold in ECC-1 (FIG. 8A) and Ishikawa cells (FIG. 9A) in response to 2 mM dibutyryl-cyclic AMP (cAMP), by ˜4 fold with 50 μM forskolin, but were unchanged by 100 Units/ml human chorionic gonadotropin (hCG), 10 nM progesterone, 10 nM dihydrotestosterone (DHT) or 10 ng/ml EGF. The increase in MAGE-11 mRNA by dibutyryl-cAMP was dose dependent as shown for Ishikawa cells (FIG. 9A). Pretreatment with 10 nM E₂ for 48 h increased PR-B levels in both cell lines (FIG. 10A) but there was no increase in MAGE-11 mRNA with subsequent progesterone treatment (data not shown). The cAMP induced increase in MAGE-11 mRNA was inhibited by 10 nM E₂ to a greater extent in ECC-1 cells (FIG. 8B) than Ishikawa cells (FIG. 9A) possibly reflecting the higher ERα levels in ECC-1 than Ishikawa cells (FIG. 10B). The ˜67 kDa MAGE-11 protein was at higher levels in ECC-1 than Ishikawa cells (FIG. 10C, left) and declined in Ishikawa cells in response to 10 nM E₂ (FIG. 10C, right).

AR mRNA was transiently down-regulated in ECC-1 cells by 0.5 mM dibutyryl-cAMP at 0.5 to 6 h but recovered partially by 12 h (FIG. 8D) and was unchanged by 24 h (FIG. 8C). The increase in AR mRNA by 10 nM E₂ was inhibited by dibutyryl-cAMP in Ishikawa cells (FIG. 9B).

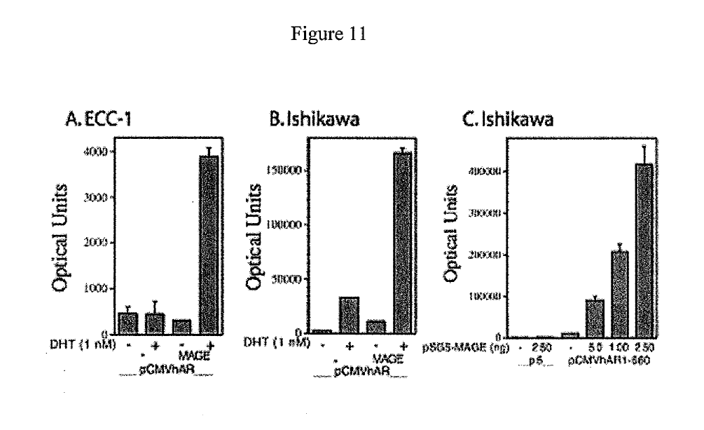

Dependence on AR transcriptional activity. The apparent disparity between increasing AR protein levels (FIGS. 3F and 3J) and declining AR mRNA levels in the secretory phase endometrium (FIG. 4C) raised the possibility that MAGE-11 influences AR stability and activity. The relatively low levels of AR and MAGE-11 in the endometrial cell lines (FIGS. 10C and 10D) required transient expression for reporter gene activation. It was found that expressing MAGE-11 strongly increased AR transcriptional activity in ECC-1 and Ishikawa cells (FIGS. 11A and 11B) and the activity of a constitutively active AR NH₂-terminal and DNA binding domain fragment in Ishikawa cells (FIG. 11C).

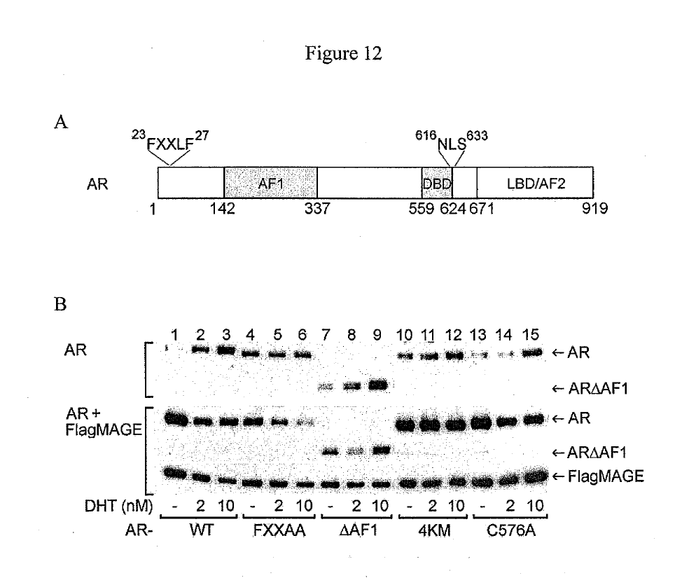

A reciprocal relationship between AR and MAGE-11 protein levels was found. In the presence of saturating DHT concentrations, AR was stabilized by the N/C interaction which was lost by an AR-FXXAA mutant (FIGS. 12A and 12B lanes 1-6) (He et al. (2000) J. Biol. Chem. 275:22986-22994; Langley et al. (1995) J. Biol. Chem. 270:29983-29990; Kemppainen et al. (1992) J. Biol. Chem. 267:968-974). On the other hand, with absent or low levels of androgen, AR is stabilized by MAGE-11 (FIG. 12B, lanes 1 and 2) which is also AR FXXLF motif dependent (lane 4) but apparently influenced, in addition, by the transcriptional status and subcellular location of AR. Transcriptionally inactive AR nuclear transport (lanes 10-12, 4KM) and DNA binding mutants (lanes 13-15, C576A) were stabilized by the N/C interaction in the presence of DHT, and by MAGE-11 in the absence and presence of DHT. However, transcriptionally inactive ARΔAF1, which lacks the NH₂-terminal AF1 activation domain, was stabilized by DHT but only weakly by MAGE-11 (lanes 7-9, ΔAF1).

In addition, it was found that MAGE-11 protein levels decline with increasing DHT concentrations depending on the transcriptional status of AR. MAGE-11 levels declined in association with wild-type AR in the presence of DHT (FIG. 12, lanes 1-3) but not with the transcriptional inactive mutants, ARΔAF1 (lanes 7-9), nuclear transport mutant 4KM (lanes 10-12) and DNA binding mutant C576A (lanes 13-15). The results suggest that the levels of AR and MAGE-11 protein are modulated in association with AR transcriptional activity.

### Discussion

MAGE-11 as AR coregulator. MAGE-11 belongs to a 12 member MAGEA gene family encoded on a 3.5 Mb segment at Xq28 of the human X chromosome (Rogner et al. (1995) Genomics 29:725-731). Before its identification as an AR coregulator, the function of MAGE-11 was unknown. Androgen binding to AR initiates a sequence of transactivation events that involves AR stabilization by the N/C interaction (He et al. (2000) J. Biol. Chem. 275:22986-22994; Langley et al. (1995) J. Biol. Chem. 270:29983-29990; Kemppainen et al. (1992) J. Biol. Chem. 267:968-974) and interaction with coregulatory proteins (Bai et al. (2005) Mol. Cell. Biol. 25:1238-1257; He et al. (2001) J. Biol. Chem. 276:42293-42301). Binding of MAGE-11 to the AR FXXLF motif relieves inhibition of coactivator binding at AF2 in the ligand binding domain imposed by the AR N/C interaction and increases recruitment of SRC/p160 coactivators that include SRC1, TIF2, and AIB1 (SRC3) (Bai et al. (2005) Mol. Cell. Biol. 25:1238-1257). These coactivators, as well as p300 and pCAF, are expressed in human endometrium during the menstrual cycle and are known to increase AR transcriptional activity (Mertens et al. (2001) Eur. J. Obstet. Gynecol. Reprod. Biol. 98:58-65; Gregory et al. (2002) J. Clin. Endocrinol. Metab. 87:2960-2966).

In the present example, evidence is provided that MAGE-11 was expressed in a temporal fashion in nuclei of the endometrial glandular epithelium from normally cycling woman. Highest levels of MAGE-11 mRNA and protein occurred during the window of receptivity to embryo implantation, increasing from a low level after ovulation to maximal levels between LH+5 and LH+10 of the menstrual cycle. The close correlation in timing of MAGE-11 expression with the window of receptivity at LH+6 through LH+10 (Psychoyos (1973) Vitam. Horm. 31:201-256; Lessey (2000) Baillieres Best Pract. Res. Clin. Obstet. Gynaecol. 14:775-788) and its localization with AR in epithelial cell nuclei provide evidence that AR and MAGE-11 have a transcriptional role in preparing the uterus for implantation and pregnancy.

The present studies using two human endometrial cell lines demonstrated a profound sensitivity of MAGE-11 expression to inhibition by estrogen. The importance of estrogen receptor mediated down-regulation in establishing the timing of receptivity to implantation (Lessey et al. (1988) J. Clin. Endocrinol. Metab. 67:334-340) is supported by αvβ3, another proposed marker of receptivity whose expression is inhibited by estrogen (Somkuti et al. (1997) J. Clin. Endocrinol. Metab. 82:2192-2197). A surprising time dependent increase in MAGE-11 mRNA was observed in endometrial cell lines cultured in phenol red free, charcoal stripped serum that was blocked by E₂. The increase in MAGE-11 mRNA was more evident in ECC-1 cells than Ishikawa cells and appeared to reflect a time dependent depletion of residual estrogenic activity in the charcoal stripped serum. This was supported by the increase in MAGE-11 mRNA by the ERα antagonist ICI-182,780 in the absence and presence of added E₂ and the even greater increase in MAGE-11 mRNA levels in serum free medium. Differences in sensitivity to estrogen inhibition of MAGE-11 expression between ECC-1 and Ishikawa endometrial cell lines correlates with ERα levels even though estrogen induced an increase in AR mRNA in both cell lines. In agreement with these findings, the concentration of E₂ required to partially inhibit MAGE-11 expression was ˜100 fold less than that required to increase AR expression. There may also be differences in autonomous second messenger signaling since cAMP reversed the inhibitory effect of E₂ to a greater extent in Ishikawa cells than ECC-1 cells.

Timing of the post-ovulatory increase in MAGE-11 in endometrial biopsies between LH+5 through LH+10 coincident with the window of receptivity to embryo implantation and the increase in MAGE-11 mRNA in response to dibutyryl-cAMP in endometrial cell lines raise the possibility that LH secreted by the pituitary acts on the endometrium to increase MAGE-11. Indeed, studies suggest direct effects of LH on the uterus, independent of the ovary, that help to prepare the endometrium for implantation (Tesarik et al. (2003) Reprod. Biomed. Online 7:59-64). Progesterone also acts directly on endometrial epithelial cell gene expression during the secretory phase and indirectly through stromal cells to induce paracrine factors (Lessey (2003) Steroids 68:809-815) such as calcitonin, a proposed marker of uterine receptivity that increases cAMP production in Ishikawa cells (Li et al. (2006) Endocrinology 147:2147-2154). The effects of cAMP are enhanced by progesterone in stromal cells (Tang et al. (1993) Endocrinology 133:2197-2203) and activin A is a component of the cAMP signaling pathway (Tierney and Giudice (2004) Fertil. Steril. 81:899-903). In support of a stromal cell effect of progesterone, a potentiating effect of progesterone on MAGE-11 mRNA levels in the endometrial cell lines was not observed. Differentiation of human endometrial stroma cells is promoted by prostaglandin-E2, LH and relaxin (Telgmann et al. (1997) Endocrinology 138:929-937), each of which increases adenylate cyclase activity and cAMP levels during the transition from proliferative to secretory stage in the human endometrium (Bergamini et al. (1985) J. Steroid Biochem. 22:299-303; Tanaka et al. (1993) J. Reprod. Fertil. 98:33-39).

The delay in maximal MAGE-11 mRNA expression until 5 days after the LH surge may reflect the reduced ERα levels of the mid-secretory phase. Loss of ERα during the mid-secretory stage would abrogate E₂ induced suppression of MAGE-11 allowing MAGE-11 levels to increase. ERα is also down regulated by progesterone in epithelial cells which correlates with the establishment of uterine receptivity (Fazleabas et al. (1999) Semin. Reprod. Endocrinol. 17:257-265; Lessey et al. (2006) Reprod. Biol. Endocrinol. 4: Suppl 1, S9 Epub ahead of print). Thus the combined actions of cAMP and E₂ appear to coordinately regulate the timing of MAGE-11 expression which may ultimately modulate AR transcriptional activity at a critical period during endometrial maturation.

Androgen action in human endometrium. Compared to the well established ERα and PR transcriptional regulators in the cyclic function of human endometrium (Lessey et al. (1988) J. Clin. Endocrinol. Metab. 67:334-340), relatively little is known about AR. The endometrium is influenced by androgens reported to circulate near constant low levels during the menstrual cycle (Jabbour et al. (2006) Endocr. Rev. 27:17-46). Evidence that AR signaling is required for embryo implantation derives from fertility defects identified in female AR knockout mice (Hu et al. (2004) Proc. Natl. Acad. Sci. USA 101:11209-11214). In agreement with previous studies in primate endometrium during the menstrual cycle, AR mRNA levels were up-regulated by estrogen in human endometrial cell lines (Slayden et al. (2001) J. Clin. Endocrinol. Metab. 86:2668-2679; Apparao et al. (2002) Biol. Reprod. 66:297-304; Narvekar et al. (2004) J. Clin. Endocrinol. Metab. 89:2491-2497; Fujimoto et al. (1994) J. Steroid Biochem. Mol. Biol. 50:137-143; Slayden and Brenner (2004) Arch. Histol. Cytol. 67:393-409; Adesanya et al. (1999) Obstet. Gynecol. 39:265-270) and transiently reduced by cAMP. AR levels were reported higher in endometrial stromal than glandular epithelial cells during the proliferative phase, persistent in stromal cells in the mid-secretory stage (LH+7 to LH+10) and lower in luminal and glandular epithelial cells late in the cycle (Mertens et al. (2001) Eur. J. Obstet. Gynecol. Reprod. Biol. 98:58-65; Lessey et al. (1988) J. Clin. Endocrinol. Metab. 67:334-340; Slayden et al. (2001) J. Clin. Endocrinol. Metab. 86:2668-2679; Apparao et al. (2002) Biol. Reprod. 66:297-304; Narvekar et al. (2004) J. Clin. Endocrinol. Metab. 89:2491-2497; Burton et al. (2003) Hum. Reprod. 18:2610-2617; Horie et al. (1992) Hum. Reprod. 7:1461-1466; Mertens et al. (1996) Eur. J. Obstet. Gynecol. Reprod. Biol. 70:11-13; Villavicencio et al. (2006) Gynecol. Oncol. 103:307-314). The opposing actions of cAMP and E₂ on AR and MAGE-11 may regulate AR action in the endometrium.

The decline in AR mRNA during the mid-secretory period when AR immunostaining increases in glandular epithelial cell nuclei supports androgen induced down regulation of AR mRNA (Quarmby et al. (1990) Mol. Endocrinol. 4:22-287). Increased levels of MAGE-11 in the early to mid-secretory period may contribute to increased AR immunostaining since AR can be stabilized by MAGE-11. The present studies in COS cells show that MAGE-11 stabilizes AR in the absence or low levels of androgen which may relate to human endometrium when tissue androgen levels are low. In the presence of androgen, a reciprocal relationship exists between AR and MAGE-11 where MAGE-11 protein is destabilized in association with AR transcriptional activity. Control in the timing of MAGE-11 expression by hormones of the menstrual cycle provides a regulatory mechanism for AR transcriptional activity within an environment of low androgen in the normal cycling endometrium of women.

AR signal amplification. For the present studies, it was proposed that the relatively low circulating testosterone levels of the human female may require AR signal amplification for gene activation. Earlier evidence was presented that AR AF2 in the ligand binding domain is evolutionarily conserved and may be functionally replaced by the evolving NH₂-terminal AF1 activation domain that provides species and tissue selectivity for gene activation (He et al. (2004) Mol. Cell. 16:425-438). MAGE-11 expression is limited to primates and could have evolved to provide a mechanism for increasing AR AF2 function that is inhibited by the AR N/C interaction. MAGE-11 could have evolved in primates to facilitate androgen action in the female reproductive tract. An extension of this is that human prostate cancer cells may commandeer MAGE-11 to increase AR transcriptional activity under conditions of low circulating androgen in men undergoing androgen deprivation therapy.

### Example 2

### MAGE-11 as a Marker and Therapeutic Target for Castration-Recurrent Prostate Cancer

### Background

AR is a ligand dependent transcription factor required for prostate cancer development and progression. AR transcriptional activity is modulated by interactions with coregulatory proteins. The recently discovered AR coregulator MAGE-11 (also referred to as MAGE-A11) was initially identified in a yeast two hybrid screen of a human testis library using an AR NH₂-terminal FXXLF motif fragment as bait. Before its identification as an AR coregulator, the function of MAGE-11 was unknown. Expression of MAGE-11 is limited to human and nonhuman primates and is absent in rats or mice. The primary function of MAGE-11 is to increase AR transcriptional activity.

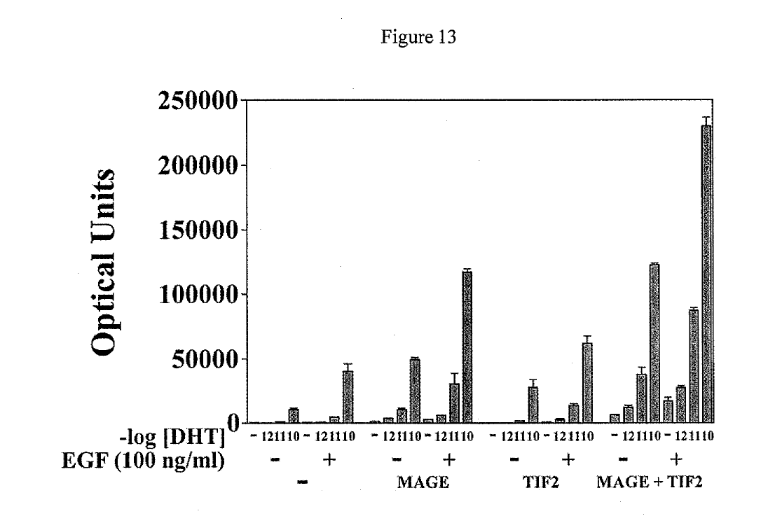

As described above, MAGE-11 binds the AR NH₂-terminal FXXLF motif to increase AR transcriptional activity by exposing activation function 2 (AF2) for increased binding of the SRC/p160 coactivators LXXLL motifs (FIG. 12A). Coexpression of MAGE-11 with transcriptional intermediary factor-2 (TIF2), an SRC/p160 coactivator, in the CWR-R1 prostate cancer cell line increased androgen dependent and independent AR transcriptional activity (FIG. 13). The increase in androgen dependent AR transcriptional activity is mediated primarily through SRC/p160 coactivator recruitment by AF2. MAGE-11 also increases AR transcriptional activity through AF1 in the AR NH₂-terminal region. Binding of MAGE-11 to the AR FXXLF motif increases AR and MAGE-11 turnover in response to growth factor signaling through the site specific phosphorylation and ubiquitinylation of MAGE-11 (Bai & Wilson E M (2008) Mol. Cell. Biol. 28, in press). In the absence of androgen, MAGE-11 is partially nuclear but colocalizes with AR in the cytoplasm where it stabilizes AR (Bai et al. (2005) Mol. Cell. Biol. 25:1238-1257). In the presence of androgen, AR and MAGE-11 colocalize in a disperse pattern throughout the nucleus.

Prostate cancer begins as an androgen dependent tumor that responds with remission to surgical or medical castration. However with time, prostate tumors regrow despite undetectable circulating androgen levels following androgen deprivation therapy. AR is almost universally expressed in all stages of prostate cancer and increased AR transcriptional activity is a hallmark of the disease (Bai & Wilson E M (2008) Mol. Cell. Biol. 28, in press). Overwhelming evidence indicates AR continues to drive prostate cancer progression. Prostate cancer cell growth is inhibited by reducing AR expression.

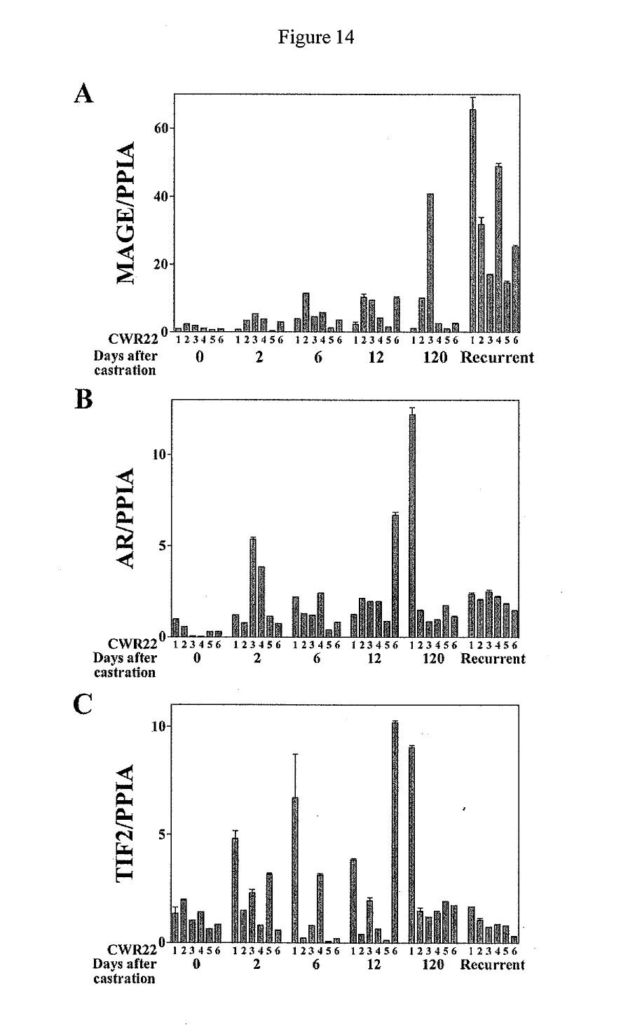

MAGE-11 mRNA levels are elevated in the LNCaP, CWR-R1 and LAPC-4 prostate cancer cell lines that express AR, but are low to undetectable in DU-145 and PC3 prostate cancer cell lines that lack AR (Bai et al. (2005) Mol. Cell. Biol. 25:1238-1257). In the CWR22 human prostate cancer xenograft, MAGE-11 mRNA levels increased 50-100 fold with transition from androgen dependence to recurrent growth in the absence of androgen (FIG. 14A). Levels of AR mRNA increased to a smaller extent with recurrent growth of the CWR22 tumor and TIF2 mRNA levels were not predictive of tumor status (FIGS. 14B and C).

The results described below show that prostate cancer recurrence is associated with increased MAGE-11 mRNA expression and that measurements of MAGE-11 mRNA levels in biopsy specimens were indicative of the extent to which prostate cancer progressed to the androgen independent state. MAGE-11 may also serve as a target for new therapeutic approaches.

### Methods

Experimental methods for MAGE-11 antibodies, immunostaining, real-time PCR, transcription assays, and immunoblot analysis were as described in Example 1.

For studies in animals, the serially transplanted androgen dependent CWR22 xenograft derived from a primary human prostate cancer and was propagated in athymic nu/nu mice to avoid tumor rejection. Animals 4-5 weeks of age were implanted subcutaneously with testosterone pellets under anesthesia using 12.5 mg sustained-release testosterone pellets placed subcutaneously in each animal by trocar. After 3-5 days, tumor cells were injected subcutaneously under anesthesia. Tumor cells were obtained from prostate cancer xenograft tumors digested with pronase. 10⁶ fresh cells were injected in 0.2 ml of Matrigel under anesthesia. Bilateral tumors grew to 0.75 g in 1-2 months and animals were castrated by scrotal incision under anesthesia. Tumors regressed following removal of testosterone pellets and castration and recurred in ˜5 months. Tumor size was monitored and tumors were not allowed to grow larger than 1 cm. Initial tumor transplantation and placement of testosterone pellets were performed under anesthesia. Animals were anesthetized using 10 μg Domitor and 1 mg Ketaset per mouse injected intraperitoneally and monitored for depth of anesthesia during surgery. Antisedan reversal (500 μg) was administered subcutaneously after surgery.

A portion of the nude male mice were castrated ˜150 days prior to sacrifice using a standard scrotal approach. Surgery to remove testes and tumor transplantation were performed under sterile conditions. After castration and removal of the testosterone pellets, Buprenorphine was administered subcutaneously (0.05 mg/kg every 12 h). Skin closures were performed with Nexabond liquid. Postoperatively mice were checked daily for complications. Survival after castration was 6-150 days before euthanasia. The procedures were in accordance with the recommendation of the American Veterinary Medical Association Panel on Euthanasia.

Animals were sacrificed by cervical dislocation after isoflurane inhalation to avoid hypoxia associated with an anesthetic overdose or CO₂, which is deleterious to tumor tissue. CWR22 tumors were not metastatic. Animal life span ranged from 2 months in noncastrated mice to 7 months for the long-term castrated animal. CWR22 tissue microarrays prepared in the ImmunoAnalysis and Tumor Management Core Laboratories of the UNC Lineberger Comprehensive Cancer Center and P01 NIH Center Grant contained 2 mm tissue cores from the androgen-dependent CWR22 human prostate cancer xenograft from intact animals and sequential time points after castration through recurrence. Formalin-fixed, paraffin-embedded tumors were used to create 60 core tissue microarrays that includes redundant time points and control tissue. For tumor transplantation, animals were bilaterally injected subcutaneously through a 22G needle.

For studies in humans, frozen tissue samples from men presenting with prostate cancer were available in the Tissue Repository of the UNC Lineberger Cancer Center, with more than 200 prostate cancer samples from African and Caucasian Americans who had undergone radical prostatectomy. These tissues were considered excess pathologic specimens routinely stored in liquid nitrogen under an exemption granted by the Institutional Review Board for use in biochemical, molecular and immunohistochemical analyses. Patients were men 35 years of age and older who varied in health status depending on the stage of prostate cancer. Men with metastatic disease may have been symptomatic from their cancer depending on tumor volume. All patient data were maintained anonymously in compliance with HIPAA guidelines. The Data and Safety Monitoring Board and the Institutional Review Board monitored all research studies for safety at the Roswell Park Cancer Institute.

Human tissue microarrays were prepared in the ImmunoAnalysis and Tumor Management Core Laboratories of the UNC Lineberger Comprehensive Cancer Center and P01 NIH Center Grant. The arrays contained more than 50 1.5 mm tissue cores from 45 men and 16 additional control cores. Androgen-stimulated benign prostate and prostate cancer cores were obtained from the transition zone of formalin-fixed, paraffin-embedded radical prostatectomy specimens from men with clinically localized prostate cancer. Selected patients did not receive radiation or hormone therapy prior to surgery. Mean age was between 46-73 years with Gleason sums of 5 to 8. Recurrent prostate cancer cores were obtained from formalin-fixed, paraffin-embedded transurethral prostatectomy specimens from men who had increasing serum prostate-specific antigen levels and urinary retention from local recurrence of prostate cancer after surgical or medical androgen deprivation therapy.

### Results and Discussion

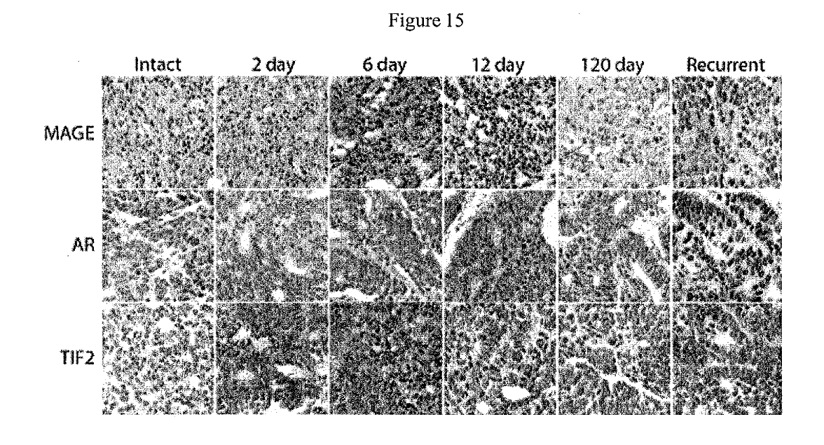

MAGE-11 expression. Parallel immunohistochemical studies using specific antibodies showed an increase in MAGE-11 protein at 6 and 12 days after castration which corresponded to the increase in MAGE-11 mRNA in the CWR22 tumor (FIG. 15). The greatest increase in MAGE-11 mRNA with the onset of castration-recurrent growth of the CWR22 tumor did not correlate with an increase in MAGE-11 protein. This may reflect increased turnover of MAGE-11 in association with increased AR transcriptional activity and mitogen signaling that increases MAGE-11 phosphorylation and ubiquitinylation. AR immunostaining increased with progression to castration-recurrent prostate cancer (FIG. 15) as previously reported (Gregory et al. (1998) Cancer Res. 58:5718-5724). TIF2 immunostaining also increased at 6 days after castration and in castration-recurrent prostate cancer which were largely independent of changes in TIF2 mRNA levels (FIG. 15). Increased SRC/p160 coactivator protein levels were previously positively correlated with prostate cancer progression (Gregory et al. (2001) Cancer Res. 61:4315-4319). The results show that increases in MAGE-11, AR and TIF protein contribute to the development of castration-recurrent prostate cancer.

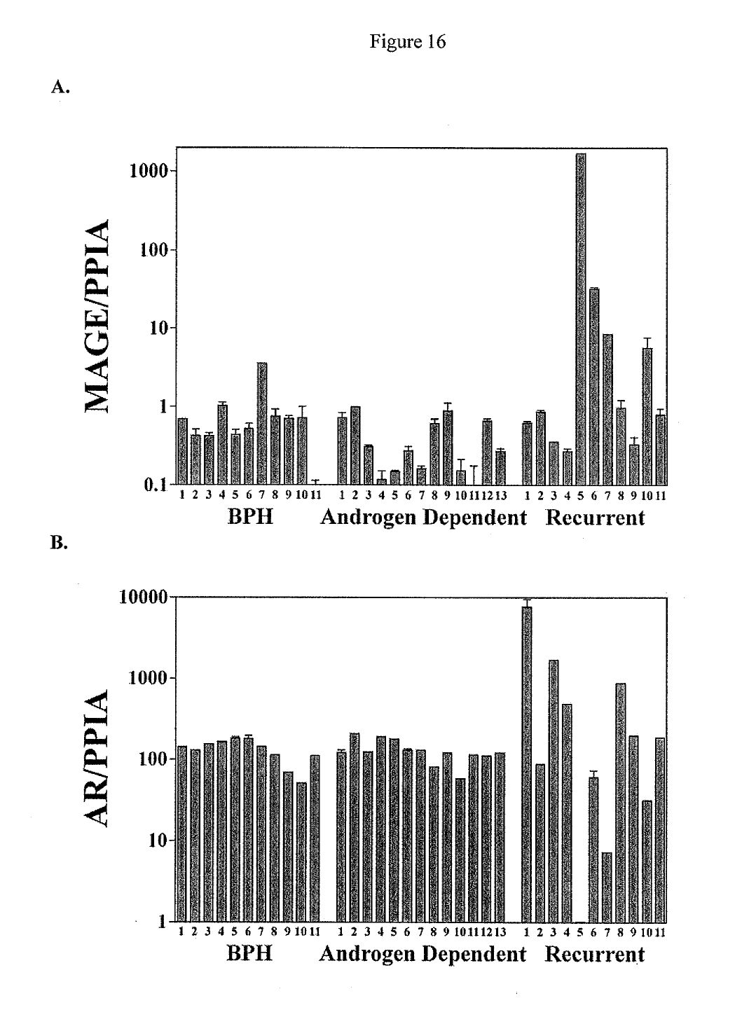

Studies using clinical specimens of benign prostatic hyperplasia (BPH) prostate, androgen dependent and castration-recurrent (androgen independent) prostate cancer specimens showed a 10 to 1000 fold increase in either MAGE-11 or AR mRNA levels in 8 out of the 11 recurrent prostate cancer samples analyzed (FIG. 16). There was an inverse relationship between AR and MAGE-11 mRNA levels in the recurrent prostate cancer samples. Recurrent prostate cancer samples with high MAGE-11 mRNA levels had lower levels of AR mRNA, and recurrent tumor samples with high AR mRNA had lower levels of MAGE-11 mRNA. In a Gleason stage 4+5=9 recurrent prostate cancer obtained 49 months after androgen deprivation therapy, there was a 1500 fold increase in MAGE-11 mRNA with clinical PSA score of 199, suggesting increased AR signaling even though AR mRNA levels were very low (FIG. 16, recurrent sample 5). The opposing increases in MAGE-11 and AR mRNA showed that there is a reciprocal relationship in regulating prostate tumor growth. The data from clinical specimens and the CWR22 xenograft of human prostate cancer showed that increased expression of MAGE-11 is indicative of prostate cancer recurrence to castration-recurrent disease and that therapeutic strategies targeting MAGE-11 may prevent prostate cancer recurrence.

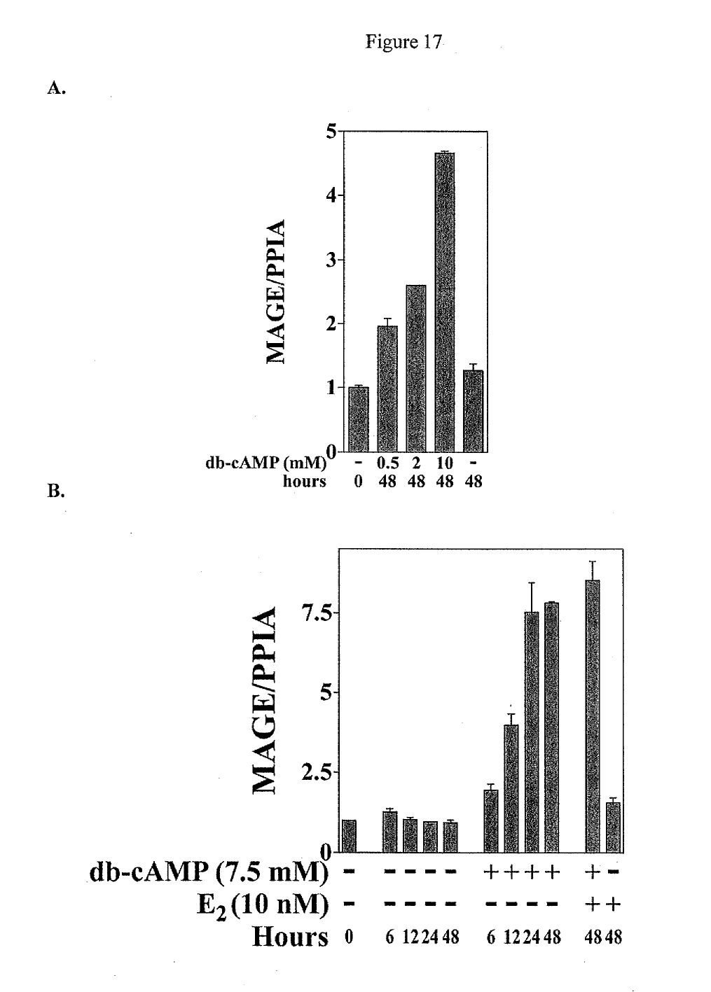

MAGE-11 gene expression is regulated by hormones in some, but not all, prostate cancer cell lines. MAGE-11 mRNA was up-regulated by cyclic-AMP in the androgen dependent LNCaP prostate cancer cell line in a dose (FIG. 17A) and time dependent manner (FIG. 17B). Induction of MAGE-11 mRNA in LNCaP cells required at least 6 h, implying a genomic effect, and was not inhibited by 17β-estradiol. MAGE-11 mRNA was also up-regulated by cyclic AMP in LNCaP-C4-2 cells, a LNCaP-derived prostate cancer cell line that is less dependent of androgen for growth. In the CWR-R1 cell line derived from the castration-recurrent CWR22 prostate cancer xenograft, MAGE-11 mRNA was not regulated by cyclic AMP, showing that regulation of the MAGE-11 gene differs between prostate cancer cell lines and may be related to the transition to castration-recurrent tumor growth.

MAGE-11 as a therapeutic target. The increase in MAGE-11 expression during prostate cancer progression in the absence of androgen shows that MAGE-11 may contribute to tumor growth through its function as an AR coregulator. MAGE-11 is both a cytoplasmic and nuclear protein and a member of the cancer-testis antigens considered to be important therapeutic targets for immune therapy in cancer treatment. Cancer-testis antigens are displayed on the cell surface in association with the integral membrane class I major histocompatibiilty complex (MHC) and are recognized by T-cells receptors which leads to destruction by killer T cells. Cancer testis antigens are therefore targets for vaccine immunotherapy because their presentation on the cell surface by the class I MHC complex elicits the T cell immune response. Cancer testis antigens are potential vaccine targets because they can induce strong spontaneous immunogenicity in humans. A number of ongoing clinical trials are currently being performed to establish the effectiveness of specific peptide, protein, DNA and RNA as vaccine therapies to induce the formation of high affinity killer T cells effective with the naturally expressed tumor antigen (Simpson et al. (2005) Nat. Rev. Cancer 5:615-625).

One of the striking findings of the present studies was that MAGE-11 protein was increased in the human prostate cancer xenograft tumor CWR22 during the period of regression following androgen withdrawal (FIG. 15). The evidence suggests that in the absence of androgen, MAGE-11 is bound to AR and stabilizes AR in the absence of androgen activation which may provide a mechanism to maintain AR expression during progression to recurrent growth of the tumor. The immunohistochemical data show that the level of MAGE-11 protein increased between 6 and 12 days in the CWR22 xenograft, showing that MAGE-11 may be a target for vaccine therapy to block progression to the recurrent state. This association with AR shows that methods that target MAGE-11 could be a useful therapeutic adjunct to androgen withdrawal therapy to prevent castration-recurrent growth of prostate cancer.

The MAGE-11 gene has three 5′ exons that code for protein sequence unique to MAGE-11 that have been shown to result in the formation of antibodies specific for MAGE-11 (Bai et al. (2005) Mol. Cell. Biol. 25:1238-1257; Bai et al. (2007) Mol. Hum. Reprod., Dec. 11 [Epub ahead of print]). In contrast, the carboxyl-terminal region of MAGE-11 is highly homologous to other members of the MAGE-11 family. The unique NH₂-terminal sequence of MAGE-11 provides immunogenic peptides that render MAGE-11 a target for active and passive cancer immunotherapeutic strategies to block progression to recurrent growth of prostate cancer. The NH₂-terminal region of MAGE-11 is therefore an immunogenic target for the destruction of prostate cancer cells as they progress to castration-recurrent growth in the absence of circulating androgen. However, it remains to be established whether MAGE-11 is expressed as a surface antigen characteristic the MAGE gene family and whether circulating antibodies are induced in CWR22 tumor bearing mice or in patients with prostate cancer. The expression of MAGE-11 and other cancer testis antigens was originally thought to be restricted to the testis and cancer. However, it has been shown that MAGE-11 is expressed in several normal tissues of the male and female reproductive tracts. Tissue selective expression of MAGE-11 nevertheless allows it to serve as a vaccine target for prostate cancer therapy.

Demethylation of the MAGE-11 gene. There is considerable evidence that the expression of the MAGE gene family members contributes to the malignant phenotype (Simpson et al. (2005) Nat. Rev. Cancer 5:615-625). MAGE-11 may not be involved in the initiation of prostate cancer but may function as an important AR coregulatory protein during prostate cancer progression to stabilize AR and increase transcriptional activity in the absence and presence of low levels of androgen. Many cancer testis genes are encoded on the X chromosome and have methylated CpG islands in normal somatic tissues that become activated by demethylation during spermatogenesis in the testis (Simpson et al. (2005) Nat. Rev. Cancer 5:615-625). The MAGE-11 gene promoter may be methylated in most normal tissues. The temporal expression of MAGE-11 in human endometrium during the menstrual cycle suggests that the MAGE-11 gene becomes demethylated and is up-regulated by hormones. Results described herein show that MAGE-11 mRNA levels are up-regulated in human endometrium by cyclic AMP and strongly suppressed by 17β-estradiol. Highest levels of MAGE-11 coincide with the window of receptivity to embryo implantation (Bai et al. (2007) Mol. Hum. Reprod., Dec. 11 [Epub ahead of print]) showing a role for AR and MAGE-11 in female fertility.

The MAGE-11 gene becomes demethylated with progression to castration-recurrent prostate cancer, which may account in part for the increase in MAGE-11 expression after prostate cancer recurrence. Demethylation occurs on a 3′ CpG island in the MAGE-11 gene promoter. Methylation of CpG islands within promoter regions is widely recognized as a mechanism to silence gene expression in normal cells (Jones & Baylin (2007) Cell 128:683-692) and may account for the low levels of MAGE-11 in most normal tissues of the male and female reproductive tracts. While many genes are silenced by DNA promoter methylation in cancer, demethylation of the MAGE-11 gene appears to represent a mechanism for increased expression in prostate cancer. Such findings are in line with previous evidence that the MAGE cancer testis antigen gene family is epigenetically regulated by hypo- or hypermethylation of the promoter (Karpf (2006) Epigenetics 1:116-120). The data show that the use of epigenetic drugs to repress MAGE-11 expression may be useful in the treatment of prostate cancer.

Hypomethylation alone is apparently not sufficient for cancer testis antigen gene expression (Simpson et al. (2005) Nat. Rev. Cancer 5:615-625). A combination of hypomethylation and hormone regulation appears to establish the increase in MAGE-11 expression in castration-recurrent prostate cancer. Timing of the onset of castration-recurrent prostate cancer after androgen deprivation in the CWR22 human prostate cancer xenograft model requires ˜120 days. This suggests acquisition of recurrent tumor growth in the absence of androgen does not result from random mutations but from a sequence of events initiated by the loss of circulating androgen. Thus, while the onset of androgen dependent cancer may be dependent on the accumulation of genetic mutations in stem cells, changes in MAGE-11 gene expression is controlled in a timed sequence initiated by androgen deprivation. The common recurrence of prostate cancer growth suggests a genetic program mediated through the AR that is initiated by androgen withdrawal.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

That which is claimed:

1. A method for detecting endometrial receptivity to embryo implantation in a female human or nonhuman primate, said method comprising the steps of:
a) obtaining an endometrial tissue sample from said primate;
b) detecting the level of expression of MAGE-11 in said endometrial
tissue sample; and
c) correlating the level of expression of MAGE-11 in said endometrial tissue sample with endometrial receptivity to embryo implantation.

2. The method of claim 1, wherein the method comprises detecting expression of MAGE-11 in endometrial tissue samples obtained from a plurality of stages of the menstrual cycle of said primate.

3. A method for monitoring endometrial maturation in a primate, said method comprising the steps of:
a) obtaining an endometrial tissue sample from said primate;
b) detecting expression of MAGE-11 in said endometrial tissue sample;
c) repeating steps a) and b) with endometrial tissue samples obtained from a plurality of stages of the menstrual cycle of said primate; and
d) correlating the level of expression of MAGE-11 in one or more tissue samples of step c) with endometrial maturation.

4. A method of in vitro fertilization in a primate, said method comprising the steps of:
a) obtaining an endometrial tissue sample from said primate;
b) detecting expression of MAGE-11 in said endometrial tissue sample;
c) repeating steps a) and b) with endometrial tissue samples obtained from a plurality of stages of the menstrual cycle of said primate;
d) correlating the level of expression of MAGE-11 in one or more tissue samples of step c) with endometrial maturation; and
e) introducing an embryo into the uterus of said primate when said endometrium is mature.

5. The method of claim 4, said method further comprising monitoring said embryo for implantation.

6. The method of claim 5, wherein said embryo develops from a zygote formed by the combination of an egg and sperm in vitro.

7. A method for diagnosing infertility in a primate, said method comprising the steps of:
a) obtaining an endometrial tissue sample from said primate;
b) detecting expression of MAGE-11 in said endometrial tissue sample;
c) repeating steps a) and b) with endometrial tissue samples obtained from a plurality of stages of the menstrual cycle of said primate; and
d) correlating delayed, reduced, increased, or early expression of MAGE-11 in one or more tissue samples of step c) with infertility in said primate.

8. The method of any of claims 1 to 7, wherein the level of expression of MAGE-11 is detected at the protein level using at least one antibody that specifically binds MAGE-11, wherein said sample is contacted with said antibody and the binding of said antibody to MAGE-11 is detected.

9. The method of claim 8, wherein detecting the level of expression of MAGE-11 comprises performing immunohistochemistry.

10. The method of any of claims 1 to 7, wherein the level of expression of MAGE-11 is detected at the nucleic acid level, wherein nucleic acid material from said sample is isolated and mixed with at least one pair of MAGE-11 primers and a thermostable DNA polymerase under conditions that are suitable for amplification by polymerase chain reaction (PCR), further wherein PCR is performed and PCR amplification products are detected.

11. The method of claim 10, wherein PCR comprises RT-PCR.

12. The method of any of claims 1 to 7, wherein the sample is obtained non-surgically.

13. The method of claim 12, wherein the sample is obtained by a uterine washing or by a uterine brushing.

14. The method of any of claims 1 to 7, wherein the sample is obtained surgically.

15. The method of any of claims 2 to 7, wherein said primate is human and the stages of the menstrual cycle are selected from the group consisting of the early secretory phase and the mid-secretory phase.

16. The method of any of claims 2 to 7, wherein said primate is human and the expression of MAGE-11 is detected on days 15 to 24 of the menstrual cycle of said human.

17. The method of any of claims 2 to 7, wherein said primate is human and the expression of MAGE-11 is detected on days 20 to 24 of the menstrual cycle of said human.

18. The method of any of claims 2 to 7, wherein said primate is human and the expression of MAGE-11 is detected on days LH+5 to LH+10 of the menstrual cycle of said human.

19. A method for detecting castration-recurrent prostate cancer in a male patient, said method comprising the steps of:
a) obtaining a prostate tissue sample from said patient;
b) detecting the level of expression of MAGE-11 in said prostate tissue sample; and
c) correlating the level of expression of MAGE-11 in said prostate tissue sample with the presence of castration-recurrent prostate cancer.

20. The method of claim 19, wherein the level of expression of MAGE-11 is detected at the protein level using at least one antibody that specifically binds MAGE-11, wherein said sample is contacted with said antibody and the binding of said antibody to MAGE-11 is detected.

21. The method of claim 20, wherein detecting the level of expression of MAGE-11 comprises performing immunohistochemistry.

22. The method of claim 19, wherein the level of expression of MAGE-11 is detected at the nucleic acid level, wherein nucleic acid material from said sample is isolated and mixed with at least one pair of MAGE-11 primers and a thermostable DNA polymerase under conditions that are suitable for amplification by polymerase chain reaction (PCR), further wherein PCR is performed and PCR amplification products are detected.

23. The method of claim 22, wherein PCR comprises RT-PCR.

24. A method for stimulating an immune response in a male patient in need thereof, said method comprising administering a human MAGE-11 protein or fragment thereof to said patient.

25. A method for treating castration-recurrent prostate cancer in a male patient in need thereof, said method comprising administering a human MAGE-11 protein or fragment thereof to said patient.

26. The method of claim 24 or 25, wherein said MAGE-11 fragment comprises the amino acid sequence set forth in SEQ ID NO:1, SEQ ID NO:2, or SEQ ID NO:3.

27. A method for inhibiting the growth of castration-recurrent prostate cancer cells in a male patient in need thereof, said method comprising contacting said cells with an agent that inhibits MAGE-11 function.

28. A method for treating castration-recurrent prostate cancer in a male patient in need thereof, said method comprising administering an agent that inhibits MAGE-11 function to said patient.

29. The method of claim 27 or 28, wherein agent that inhibits MAGE-11 function is an siRNA, an miRNA, an antisense RNA, an antisense DNA, or an antagonist of the MAGE-11 protein.

30. The method of claim 29, wherein said antagonist of the MAGE-11 protein is an antibody that specifically binds to human MAGE-11 protein or fragment thereof.

31. The method of claim 30, wherein said MAGE-11 fragment comprises the amino acid sequence set forth in SEQ ID NO:1, SEQ ID NO:2, or SEQ ID NO:3.

32. The method of claim 31, wherein said antibody is MagAb94-108, MagAb59-79, MagAb13-26 or Flag-MagAb

33. A polyclonal antibody that specifically binds to human MAGE-11 protein or fragment thereof, wherein said antibody is MagAb94-108, MagAb59-79, MagAb13-26 or Flag-MagAb.

34. A kit comprising at least one antibody that specifically binds to human MAGE-11 protein or fragment thereof and instructions for use, wherein said antibody is MagAb94-108, MagAb59-79, MagAb13-26 or Flag-MagAb.

35. The kit of claim 34, said kit further comprising instructions for using the MagAb94-108, MagAb59-79, MagAb13-26 or Flag-MagAb antibody within a method for detecting endometrial receptivity to embryo implantation in a human female.

36. The kit of claim 34, said kit further comprising instructions for using the MagAb94-108, MagAb59-79, MagAb13-26 or Flag-MagAb antibody within a method for monitoring endometrial maturation in a human female.

37. The kit of claim 34, said kit further comprising instructions for using the MagAb94-108, MagAb59-79, MagAb13-26 or Flag-MagAb antibody within a method of in vitro fertilization in a human female.

38. The kit of claim 34, said kit further comprising instructions for using the MagAb94-108, MagAb59-79, MagAb13-26 or Flag-MagAb antibody within a method for diagnosing infertility in a human female.

39. The kit of claim 34, said kit further comprising instructions for using the MagAb94-108, MagAb59-79, MagAb13-26 or Flag-MagAb antibody within a method for detecting castration-recurrent prostate cancer in a human male.

40. The kit of claim 34, said kit further comprising instructions for using the MagAb94-108, MagAb59-79, MagAb13-26 or Flag-MagAb antibody within a method for treating castration-recurrent prostate cancer cells in a human male.


SEQUENCE LISTING (6 sequences)

SEQ ID NO: 1 - protein, 15 aa, Homo sapiens
ITQIFPTVRPADLTR

SEQ ID NO: 2 - protein, 21 aa, Homo sapiens
DLPRVQVFREQANLEDRSPRR

SEQ ID NO: 3 - protein, 14 aa, Homo sapiens
SPASIKRKKKREDS

SEQ ID NO: 4 - DNA, 18 bp, Artificial Sequence
  oligonucleotide primer
tggtgctgaggattggca

SEQ ID NO: 5 - DNA, 19 bp, Artificial Sequence
  oligonucleotide primer
tagcgtgtcgaccccattc

SEQ ID NO: 6 - DNA, 24 bp, Artificial Sequence
  oligonucleotide primer
tgcccattcctatgccatcgtgtg